(19) United States
(12) Patent Application Publication
(10) Pub. No.: US 2004/0208883 A1
(43) Pub. Date: Oct. 21, 2004

(54) Method for preparing ester linked peptide-carbohydrate conjugates

(76) Inventors: Benjamin Guy Davis, Oxford (GB); Anthony John Fairbanks, Oxford (GB)

Correspondence Address: NIXON & VANDERHYE, PC, 1100 N GLEBE ROAD, 8TH FLOOR, ARLINGTON, VA, 22201-4714, US

(21) Appl. No.: 10/486,349

(22) PCT Filed: Aug. 12, 2002
(86) PCT No.: PCT/GB02/03704

(30) Foreign Application Priority Data
Aug. 10, 2001 (GB) 0119665.8

(51) Int. Cl. A61K 39/00; C12P 21/06

(52) U.S. Cl. 424/185.1; 435/68.1; 530/395

(57) ABSTRACT

A method of producing an ester linked carbohydrate-peptide conjugate is provided comprising: (a) providing a vinyl ester amino acid group, and (b) reacting the vinyl ester amino acid with a carbohydrate acyl acceptor in the presence of an enzyme, to produce thereby an ester-linked carbohydrate-peptide conjugate. Also provided are ester linked carbohydrate-peptide conjugates obtainable by such methods.

### FIELD OF THE INVENTION

The present invention relates to the preparation of peptide-carbohydrate conjugates and in particular to the synthesis of ester linked peptide-carbohydrate conjugates.

### BACKGROUND TO THE INVENTION

Many carbohydrate-peptide conjugates display a wide variety of potent biological activities of potential therapeutic and commercial value (Davis (1999) and Taylor (1998)). For example, glycoproteins act as critical cell surface communication markers (Varki (1993)), glycopeptide motifs such as the Thomsen-Friedenreich (Tf) antigen are associated with cancer cell lines (Springer (1997)) and an oligomeric sequence of the glycopeptide motif (AAT[Galβ(1,3)GalNAcα])ₙ displays unusual non-colligative antifreeze properties (Tsuda and Nishimura (1996)). Access to well-defined carbohydrate-peptide conjugates and their analogues to probe the nature of these properties is essential. A large number of elegant methods have been developed for the synthesis and assembly of N- and O-linlked glycopeptides (Taylor (1998) and Seitz (2000)) but these methods may be complicated by low glycosylation efficiencies and often require the use of extensive protection regimes to ensure regioselectivity. To avoid these potential problems and with the ongoing goal of finding a rapid and efficient method of linling carbohydrates to amino acids to construct ester-conjugated glycopeptides (Tennant-Eyles and Fairbanks (1999)) we have investigated the utility of enzyme-catalyzed regioselective acylation of carbohydrates as a one-step method. Several biofunctional molecules, such as enkephalin-carbohydrate conjugates that modulate fibroblast and melanoma growth, are themselves β-amino esters of carbohydrates. Moreover, carbohydrate-peptide conjugates connected by potentially metabolisable, sacrificial linkages, such as esters, have high potential utility as prodrugs in which the glycan moiety affords both protection and specific transport properties.

### SUMMARY OF THE INVENTION

The protease-catalyzed synthesis of amino acid ester-carbohydrate conjugates as glycopeptide analogues has been achieved in a highly regioselective and carbohydrate-specific manner using amino acid vinyl ester acyl donors and minimally or completely unprotected carbohydrate acyl acceptors. Together these probed active sites of proteases to reveal yield efficiencies that are modulated by the carbohydrate C-2 substituent. This may be exploited to allow selective one-pot syntheses.

Thus, in accordance with the present invention, there is provided a method of producing an ester linced carbohydrate-peptide conjugate comprising:

(a) providing a vinyl ester amino acid group, and

(b) reacting the vinyl ester amino acid with a carbohydrate acyl acceptor in the presence of an enzyme such as a protease, to thereby produce an ester-linked carbohydrate-peptide conjugate.

In preferred aspects, step (a) comprises protecting the amine group of the amino acid with a protecting group, and reacting the amino acid with vinyl acetate to produce the vinyl ester amino acid group. The vinyl ester amino acid group may be vinyl ester phenylalanine. The amino acids can be extended by terminal chain extension to produce a desired peptide. The acyl carbohydrate acceptor may be unprotected. Preferably the conjugate has 6 O-regioselectivity. In preferred aspects, the carbohydrate acyl acceptor is D-mannose. Sugar reducing extensions can also be carried out to provide a desired carbohydrate.

Carbohydrate-peptide conjugates produced in accordance with the present invention can be formulated with a pharmaceutically acceptable carrier, for delivery for administration to an individual in need thereof.

### DESCRIPTION OF THE FIGURES

FIG. 1 shows schemes 1, 2 and 3 for reactions described in more detail in Example 1.

FIGS. 2 to 5 show schemes 4 to 13 for reactions described in more detail in Example 2.

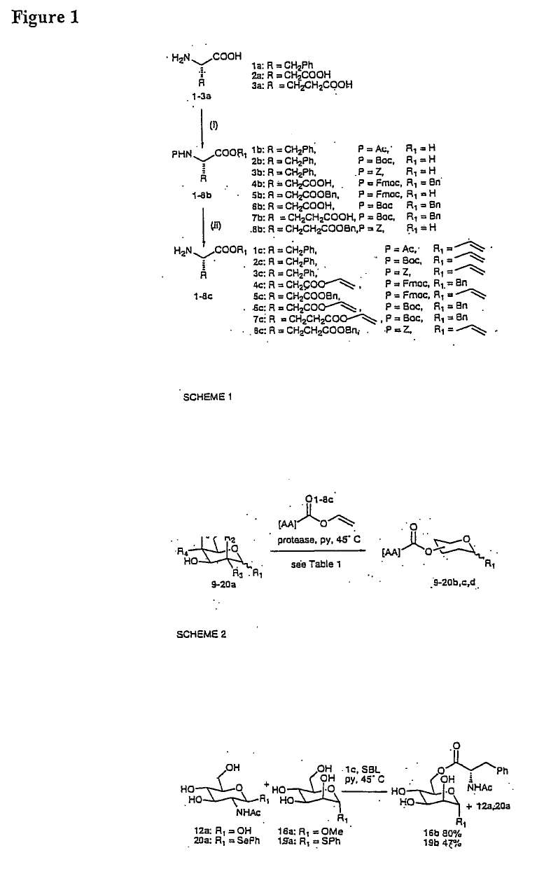

### DETAILED DESCRIPTION OF THE INVENTION

The present invention provides a method for synthesising peptide-carbohydrate conjugates. In particular, the invention uses amino acid vinyl ester acyl donors and carbohydrate acyl acceptors in the presence of an enzyme such as a protease.

In accordance with one aspect of the invention, a process for synthesising a peptide-carbohydrate conjugate is provided by enzyme catalysed acylation. An amino acid vinyl ester is reacted with a carbohydrate acyl acceptor in the presence of a enzyme such as a protease to thereby produce the desired peptide-carbohydrate conjugate.

In accordance with one aspect of the invention, the conjugation reaction is carried out using an amino acid vinyl ester. The amino acid vinyl ester may be provided by protecting the amine (NH₂) group of the selected amino acid and subsequently reacting the amino acid with vinyl acetate or other vinyl compounds. Any suitable protecting group may be used such as amine and ester protecting groups such as Ac, Boc, Fmoc, Z or Bn. Subsequently, the protecting groups can be removed. The removal of the protecting group may be at any suitable point. In some embodiments the protecting group may be removed prior to the conjugation of the vinyl ester amino acid to the carbohydrate. In other embodiments the protecting group may be removed after the conjugation of the vinyl ester amino acid with the carbohydrate.

The removal of a protecting group may allow the addition of other molecules or groups to the conjugate. For example, removal of a group protecting an amino group may allow the addition of amino acids or peptides to the conjugate to extend the peptide part of the conjugate. It may also allow the addition of bridging molecules such as, for example, diethyl squarate. This may allow several peptide carbohydrates conjugates to be joined together. Protecting groups may be removed from several conjugates to reveal reactive groups on each, such as amino groups, and the conjugates may then be reacted with the groups on the bridging molecule to join them together.

In some embodiments of the invention, more than one protecting group may be present. In some cases different protecting groups will be present in a conjugate, with each type of protecting group being removable under different conditions. This may allow selective stepwise deprotection with modifications being made to the conjugate between deprotections.

Any suitable amino acid vinyl ester may be used in the invention. Vinyl esters of any of the naturally occurring amino acids such as those of alanine, cysteine, aspartic acid, glutamic acid, phenylalanine, glycine, histidine, isoleucine, lysine, leucine, methionine, asparagine, proline, glutamine, arginine, serine, threonine, valine, tryptophan or tyrosine may be employed. Vinyl esters of derivatives of such amino acids or artificial amino acids may also be used in the invention. In a preferred embodiment of the invention phenylalanine vinyl ester may be employed as the vinyl ester amino acid. In another preferred embodiment of the invention a vinyl ester of an acidic amino acid such as, for example, aspartic acid vinyl ester or glutamic acid vinyl ester may be employed and in particular glutamic acid vinyl ester may be employed. In some embodiments of the invention the vinyl amino acid ester may have multiple vinyl ester groups which each can be used to conjugate a carbohydrates to.

Any suitable carbohydrate acyl acceptor may be used in accordance with the invention. Preferably, carbohydrates are selected to produce peptide-carbohydrate conjugates of desired regioselectivity taking into account the amino acid vinyl ester acyl donor and catalytic enzyme used in the reaction. Examples of suitable carbohydrates include saccharide subunits such as mannose, glycose, galactose, acetyl-D-glucose, and riboses such as nucleotides or nucleosides, such as adenosine or uridine. D-gluco sugars in particular are preferred. These carbohydrates can be reacted in the deprotected state, in general, leading to 6-O regioselectivity.

In a preferred aspect of the invention, the carbohydrate is provided in deprotected form. Alternatively, carbohydrates may be protected using anomeric substituants, for example, methyl groups at O-1. In one aspect of the invention thioglycosides or selenoglycosides may be used. Other protecting groups may be used to achieve alternative regioselectivity. Modulation of the sugar ring substituents, such as the C2 substituent of the sugar of the carbohydrate for conjugation can also be carried out to obtain a desired regioselectivity.

Preferred enzymes to catalyse the reactions of the present invention are proteases, and in particular, serine proteases. Thermolysin or bacterial subtilisins, such as subtilisin of bacillus lentus (SBL) are preferably used. Other enzymes such as esterases, acylases or lipases may also be used. Enzymes may be employed in the form of cross-linked enzyme crystals (CLECs) and this may, in particular, be the case for thermolysin. The enzyme employed may be chosen on the basis of the identity of the vinyl ester amino acid as certain enzymes may show optimal activity with a particular vinyl ester amino acid group. For example, thermolysin may be preferably employed where the vinyl ester amino acid used is an acidic amino acid such as aspartic acid or glutamic acid. For conjugations involving phenylalanine vinyl ester the preferred enzyme may be a subtilisin and in particular SBL.

The acylation reaction may be carried out under any suitable conditions. In general, conditions will be selected based on the particular enzyme being used to catalyse the reaction. Typically, reactions are carried out in the temperature range of 20° C. up to 60° C. depending on the enzyme. However, lower or higher temperatures could be used depending on the optimum temperature for the selected enzyme. For some enzymes higher reaction temperatures may be employed, such as a temperature from 50 to 75 C., preferably from 55 to 70° C. and more preferably from 60 to 65° C. Conjugations involving acidic amino acids vinyl esters such as, for example, aspartic acid vinyl ester or glutamic acid vinyl ester, may employ such higher temperatures and this may be the case, in particular, where the enzyme catalysing the reaction is thermolysin.

Acylation reactions are generally carried out under mild conditions. The reactions may be carried out in organic solvents, preferably, dry organic solvents such as DMF, pyridine, N-methylmorpholine, tert-butanol. The acylation reactions are carried out over a suitable time course, which may be selected based on the temperature of the reaction, quantity of enzyme present and rate of conjugation for the selected peptide-carbohydrate. The particular enzyme employed may also influence the reaction time. Typically a reaction may be carried out over 20 to 800 hours, such as from 50 to 500 hours, preferably from 100 to 400 hours and more preferably from 100 to 200 hours. Samples may be talcen from the reaction vessel to determine the presence of the desired product and monitor the progress of the reaction.

Some reactions may give optimal yields, or particular products, if fairly long reaction times are employed such as, for example, from two to five weeks, preferably from 10 to 28 days, and more preferably from 14 to 21 days. For example, reactions involving ribonucleotides such as adenosine or uridine ribonucleotides as the carbohydrate acyl acceptors may employ such longer reaction times. In some embodiments of the invention conjugations involving such longer incubation times may also employ higher temperatures such as from 50 to 75° C., preferably from 55 to 70° C. and more preferably from 60 to 65° C. In particular these higher temperatures and reaction times may be employed when thermolysin is the enzyme employed and/or the vinyl ester amino acid is aspartic acid vinyl ester or glutamic acid vinyl ester.

After the acylation reaction, any protecting groups on the amino acids may be removed by any suitable technique known to those skilled in the art. For example, Boc may be removed by trifluoroacetic acid, Z by hydrogenation with Pd. BOC may be deprotected using AcCl and MeOH or combination of Et₃ S₁H and DCM. In addition, an enzyme-cleavable system may be used, for example, using phenacetyl PhCH₂C(O)-protection on nitrogen and deprotection by the action of the enzyme penicillin G acylase under mild conditions. Carbohydrate residues that have been protected, for example, using anomeric substituants can also be deprotected by suitable methods well-known to those skilled in the art.

In one aspect of the present invention, the amino acid vinyl ester group may be provided as part of a peptide chain, such as a di- or tri-peptide. In all aspects of the present invention, chain extension reactions may be carried out to either the peptide portion or the carbohydrate portion of the conjugate produced by the acylation reaction. Thus, the conjugation step may be used to provide a building block for extension to produce any desired peptide-carbohydrate conjugate.

In one aspect of the present invention, the amino acid or peptide group is deprotected following acylation to yield a free amino terminus. Subsequently, peptide coupling may be carried out using conventional techniques to yield the desired peptides. For example, Boc can be removed by incubation with hydrochloric acid and methanol. Peptide coupling can be carried out using water-soluble carbodiimide with a peptide to extend the peptide chain. The peptide added to the conjugate may, for example, be from two to twenty amino acid residues in length, preferably from three to fifteen residues and more preferably from five to ten residues in length. The peptide may be longer such as from twenty to forty amino acids. In some embodiments a single amino acid may be added or amino acids or peptides may be added sequentially to gradually increase the length of the chain.

Sugar reducing end extension may also be carried out to extend the carbohydrate portion of the conjugate. Thus, similarly, the carbohydrate acyl acceptor used in the acylation reaction may provide a building block for provision of a selected carbohydrate group. Preferably, the amino acid of the conjugate is provided and retained in a protected form while carrying out sugar derivatization of the conjugate. Subsequently, protecting groups may be removed as described above.

In a particularly preferred example of the present invention, phenylalanine is used as the amino acid for conjugation to N-mannose in the presence of a serine protease such as subtilisin. This particular reaction demonstrates 6-O regioselectivity using unprotected D-mannose. This conjugation leads to specific 6-O regioselectivity and can be used to enable production of any desired peptide-carbohydrate conjugate through extension of the amino acid chain and/or extension of the carbohydrate chains. In addition, thio or seleno mannosides may be used as the acyl acceptor, or other O-1 substituents of mannose, to improve the yield obtained.

The enzyme catalysted acylation of the present invention allows production of carbohydrate-peptide conjugates which can be used as a basic building block to generate desired carbohydrate-peptide conjugates having a desired biological activity. The sugar or peptide extension reactions described above can be used either to provide a desired carbohydrate-peptide conjugate having a desired biological activity. The carbohydrate-peptide conjugates of the present invention may also be provided in which the peptide has a selected physiological activity and in which the sugar portion of the conjugate is used to protect and/or target the peptide to a particular location. In the alternative, the peptide portion of the conjugate may be used to protect and/or target a physiologically active carbohydrate to a desired location. Preferably, the ester linkage is degraded through enzymatic action or other suitable conditions to release the peptide or sugar physiologically active agent respectively. The peptide region may contain the recognition sequence for a particular protease, allowing part of the peptide to be released from the conjugate following hydrolysis. In one embodiment of the invention, the peptide may be chemotactic for certain cells and in particular immune cells such as, for example, macrophages and/or neutrophils. The chemotactic region of the peptide may comprise the tripeptide sequence f-Met-Leu-Phe. The chemotactic region of the peptide may be released from the conjugate enzymatically once it has been administered to a subject.

In a preferred aspect of the invention, the regioselectivity of the acylation reactions may be exploited to obtain selective conjugation of an amino acid vinyl ester to a desired sugar, in particular, where such sugar may be provided in a rmixture of sugars.

The acylation and/or acylation and extension reactions of the present invention may also be used to generate glycopeptide/glycoprotein mimics which are easier to assemble and can be used, for example, as probes of any glycoprotein interaction. In another aspect, the acylation reactions of the present application are used to create tRNA molecules. Such amino acylated ribonucleotides are central to the biosynthesis of proteins. The methods of the present invention could therefore be used to create natural and unnatural tRNA-amino acid linked ester conjugates as probes or for use in protein biosynthesis.

The generation of tRNA-amino acid conjugates can make use of the 2′/3′ over 5′ hydroxyl (secondary over primary) aminoacylation specificity seen when using the methods of the invention to conjugate ribose sugars with vinyl ester amino acids. Standard chemical methods, or the use of other enzyme systems, in ribose acylation gives 5′-O-acyl derivatives which are of no use in acyl-tRNA synthesis. However, by reacting a ribonucleotide, such as riboadenosine or ribouridine, with a vinyl ester amino acid, 2′/3′ rather than 5′ acylation is seen. Thus the methods of the invention may be used to produce 2′/3′ OH acylated ribose carbohydrates, such as acylated ribonucleotides. In particular, riboadenosine or ribouridine may be acylated. Ribose sugars with other groups in place of a base may be acylated, for example methyl ribose may be acylated.

It is thought that in these acylations that initially the 2′ OH group of the ribose is acylated followed by migration from the 2′ OH to the 3′OH. The O-3′ and O-2′ acylated products are in equilibrium and both can be used to generate amino acyl tRNAs.

Subtilisins and preferably SBL may be used to carry out the acylation of ribose acyl acceptors. Reaction temperatures for the acylation of ribose acyl acceptors may, for example, be in the range of from 30 to 60° C., preferably from 40 to 50° C. and more preferably from 43 to 47° C. Typically, the reaction may be carried out over from one to five weeks, preferably from two to four weeks and even more preferably for about three weeks. In a preferred embodiment of the invention the vinyl ester amino acid employed will be phenylalanine vinyl ester.

Following acylation of a ribonucleotide reactions may be carried out to add a further nucleotide to the 5′OH of the conjugate. For example, a subsequent regioselective (selective phosphitylation of the primary 5′OH) phosphoramidite coupling strategy and iodine mediated oxidation may be employed to generate an amino acyl dinucleotide, preferably amino acyl CA dinucleotide. This amino acyl tRNA precursor may be modified to extend the carbohydrate region of the conjugate and hence generate a full amino acyl-tRNA molecule or truncated amino acyl-tRNA mimic.

The methods of the present invention may also be employed to generate peptide-bridged carbohydrates. By using vinyl ester amino acids with more than one vinyl ester group, or by employing bridging molecules, it is possible to have two or more carbohydrate moieties present in the conjugate linked by the amino acid or bridging molecule.

Due to the regioselectivity of enzymes such as SBL it is possible to conjugate a chosen sugar to a specific ester group in the vinyl ester amino acid and then further sugars may be conjugated to the other ester groups in the vinyl ester amino acid. This removes the need for complicated protection strategies when generating bridged conjugate with a specific structure. For example, the vinyl ester amino acid may have a phenylalanine vinyl ester group at one end and an orthogonal vinyl ester group at its other group. SBL may be used to link a sugar to the phenylalanine vinyl ester group and a second enzyme, such as TL-CLEC, may be used to conjugate a different sugar to the orthogonal group.

In further aspects of the present invention, a desired peptide-carbohydrate conjugate prepared in accordance with the present invention is formulated together with a pharmaceutically acceptable carrier for subsequent administration to the human or animal body.

The invention also relates to peptide-carbohydrate conjugates obtainable in accordance with the conjugation reactions of the invention.

The invention is hereinafter described in more detail with reference to the accompanying examples.

### EXAMPLE 1

Initially, we chose the serine protease subtilisin Bacillus lentus (SBL, EC 3.4.21.14) as a powerful catalyst for ester synthesis (Dickman and Lloyd (1998)) and the representative amino acids phenylalanine 1a, aspartic acid 2a and glutamic acid 3a. As Scheme 1 of the Figures illustrates, selective protection of 1-3a afforded the corresponding carboxylic acid derivatives 1-8b. These amino acid derivatives were chosen to probe not only the amino acid specificity of SBL but also its tolerance in the amino acid for a variety of amine (Ac, Boc, Fmoc, Z) and ester (Bn) protecting groups. Vinyl esters are useful acyl donors that render transesterifications essentially irreversible. Pd(OAc)₂-mediated transesterification (Lobell and Schneider (1994)) of 1-8b with vinyl acetate (Scheme 1) allowed the preparation of the corresponding Phe, Asp and Glu; a and side-chain vinyl esters 1-8c. In all cases, 1-8c showed no non-enyzmatic reaction with 9-20a.

With these acyl donors as both building blocks for glycopeptide construction and as probes of enzyme specificity in hand, we investigated their utility in transesterification reactions with a representative range of carbohydrate acyl acceptors 9a-20a (Scheme 2 of the Figures, Table 1 below). After exploring a range of conditions, the use of SBL lyophilised from phosphate buffer (pH 8.0) in anhydrous pyridine at 45° C. proved optimal. The use of other solvents (DMF, N-methylmorpholine, t-BuOH); different temperatures; SBL lyophilized in the absence of or with other buffers; alternative acyl donors (e.g. vinyl acetate); or different molar ratios (<1.6 equiv. donor gave less monoacyl, >1.6 equiv gave diacyl in some cases) resulted in transesterification but with generally lower efficiencies.

Initial variation of parent carbohydrate in the completely deprotected series 9a-12a revealed exclusive O-6 regioselectivity but only low yields of either D-glucose 9b or D-galactose 10b 6-O-phenylalaninate esters. Regiochemistry of O-X-esterification products was confirmed ¹H, ¹³C NMR e.g., 18a ¹H NMR (CD₃OD) β 3.62 (H-6), 3.66 (H-6′); ¹³C NMR (CD₃OD) β 62.6 (C-6) 18b ¹H NMR (CD₃OD) β 4.23 (H-6), 4.32 (H-6′); ¹³C NMR (CD₃OD) β 65.9 (C-6) and by some or all of HMBC, HSQC NMR experiments, acylation of remaining hydroxyl groups and OH—H cross pealcs in d₆-DMSO COSY. A higher yield of the 6-O-phenylalaninate ester of D-mannose 11b indicated an exciting preference based only on the stereochemistry of the parent carbohydrate. This crucial dependency on carbohydrate acceptor was yet more dramatically confirmed by the complete absence of product from the attempted esterification of N-acetylglucosamine 12a from which only 12a and the product of acyl donor hydrolysis 1b were recovered.

TABLE 1
Carbohydrate-Amino Acid Coupling Reactions.

| Carbo- | | | | | | | | Yield of | Yield of |
|---|---|---|---|---|---|---|---|---|---|
| hydrate Acyl | Acyl | | | | | | | 6-O-acyl | 3-O-acy |
| Acceptor | Donor | Enzyme | R₁ | R₂ | R₃ | R₄ | R₅ | (%)^{c} | (%)^{c} |
| 9a | 1c | SBL^{a} | OH | H | OH | OH | H | 24 9b | — |
| 10a | 1c | SBL^{a} | OH | H | OH | H | OH | 24 10b | — |
| 11a | 1c | SBL^{a} | OH | OH | H | OH | H | 49 11b | — |
| 12a | 1c | SBL^{a} | OH | H | NHAc | OH | H | — | — |
| 13a | 1c | SBL^{a} | α-OMe | H | OH | OH | H | 25 13b | — |
| 14a | 1c | SBL^{a} | β-OMe | H | OH | OH | H | 28 14b | — |
| 15a | 1c | SBL^{a} | β-OMe | H | OH | H | OH | 30 15b | — |
| 16a | 1c | SBL^{a} | α-OMe | OH | H | OH | H | 76 16b | — |
| 17a | 1c | SBL^{a} | β-SPh | H | OH | OH | H | 44 17b | 29 17c |
| 18a | 1c | SBL^{a} | β-SPh | H | OH | H | OH | 36 18b | — |
| 19a | 1c | SBL^{a} | α-SPh | OH | H | OH | H | 62 19b | — |
| 20a | 1c | SBL^{a} | β-SePh | H | NHAc | OH | H | 23 20b | — |
| 16a | 1c | TL- | α-OMe | OH | H | OH | H | 48 16b | — |
| | | CLEC^{b} | | | | | | | |
| 16a | 2c | SBL^{a} | α-OMe | OH | H | OH | H | 32 16c | — |
| 16a | 2c | SBL^{d} | α-OMe | OH | H | OH | H | 63 16c | 17 16d |
| 16a | 3c | SBL^{d} | α-OMe | OH | H | OH | H | 60 16e | — |

^{a}2 mg · ml⁻¹ of lyophilized (from pH 8.0, 0.1 M phosphate) enzyme preparation equiv to 0.006 mol %, 45° C., anhydrous pyridine, 120 h.
^{b}1 mg · ml⁻¹ of CLEC preparation, 45° C., 1:25 water:pyridine.
^{c}All yields are for isolated, purified, single compounds.
^{d}As for ^{a} but for 500 h

Next the effect of anomeric substituent was probed. Introduction of a methyl substituent at O-1 increased yield only slightly in the case of D-galactose and D-glucose acyl acceptors 13-15a. Moreover, the near identical yields of α- and β-glucosides 13,14b indicated that, at least in the D-gluco series, anomeric stereochemistry had little or no effect on overall yield. Most notably, the apparent specificity preference of SBL for D-manno acyl acceptors observed in the formation of 11b was further confirmed by the again higher yield (76%) of ester 16b obtained here from α-D-mannoside 16a.

Thioglycosides and selenoglycosides are important glycosyl donors (Davis (2000)) and we next investigated their esterification to provide potential glycopeptide donors, in which the glycosyl unit might be further extended to higher oligosaccharide products after formation of the peptide-glycan linlk, and as further probes of the effect of anomeric substituent in the carbohydrate acyl acceptor. Consistent with both their larger size and the potential for aromatic aglycones in carbohydrate substrates to interact with protein surfaces (Chung and Takayama (1998)), more dramatic results were obtained for the thioglycosides 17-20a. A trend in the efficiencies of the formation of 6-O-phenylalaninate products in the order D-manno>D-gluco>D-galacto>N-acetyl-D-gluco emerged. In addition, for the first time, reduced regioselectivity was observed for D-thioglucoside 17a (3:2, 6-O 17b: 3-O 17c). The question of whether 17c is a direct or indirect, rearranged acylation product was investigated. 17b, under standard reaction conditions but in the absence of donor 1c, did not yield 17c.

Next we investigated the effect of varying the amino acid acyl donor. Consistent with the observed low affinity of SBL for other amino acid esters, none of the aspartate or glutamate acyl donors were accepted as substrates. In all cases only vinyl esters 4-8c were recovered indicating an absence of productive binding by SBL to form acyl-enzyme intermediate. This contrasted with the reactions of 1c from which only transesterification or hydrolysis products were recovered. In order to further assess the utility of 1,4-8c as acyl donor probes, we also screened their reactivity with CLEC-thermolysin (TL-CLEC-CLEC's are cross-linked enzyme crystals) as a protease with a different substrate specificity profile, that includes β-aspartate esters (Niyanaga et al., (2000)). TL-CLEC also accepted 1e allowing the preparation of 16b from 16a in 48% yield.

Next, the effect of N-protection in the acyl donor was investigated using Boc- and Z-protected phenylalanine donors 2,3c, respectively. For 2c much lower rates of reaction were observed than for 1c and after a comparable period of time lower yields (32%) for the esterification of 16a were obtained. However, extended reaction times gratifyingly allowed the preparation of 6-O-phenylalaninates 16c,e from 2,3c in 63 and 60% yields, respectively. The utility of 16c,e as glycopeptide building blocks was confirmed through their quantitative N-deprotection to methyl 6-O-phenylalaninyl-α-D-mannopyranoside 21, which may be extended at its N-terminus.

16c was deprotected with HCl, MeOH to yield a free amino terminus. Peptide compiling using EDCI, water soluble carbodiimide, with tri-peptide formyl-Met-Len-Phe-OH formed a tetrapeptide sugar, formyl-Met-Len-Phe-Phe-D-Man-α-OMe conjugate in 72% yield.

Finally, the valuable specificity information obtained in these screens was exploited to allow selective one-pot couplings. We were delighted to find that different carbohydrate acyl acceptors successfully competed in one-pot reactions to allow carbohydrate-selective esterification. Thus, in 1:1 mixtures of 12a+16a and 19a+20a (Scheme 3 of the Figures) mannosides reacted over N-acetylglucosaminides with 1c in SBL-catalyzed acylations to yield mannoside esters 16,19b exclusively. In both reactions no trace of 12b or 20b, respectively, was detected during this highly selective process.

In summary, we have described a ready method for the construction of glycan-peptide conjugates by exploiting a highly regioselective protease-catalyzed transesterification process. The yields for this selective carbohydrate-peptide linlkage construction of 23-76%, compare well with overall yields of <34% for alternative routes employing protection-deprotection strategies (Tennant-Eyles and Fairbanks (1999)). The glycopeptides formed are powerful building blocks that will allow sugar reducing end (e.g. 17-20a) or peptide N-terminal (e.g., 21) extension. In addition, we have probed the substrate specificity of the proteases SBL and TL-CLEC in this reaction using the novel vinyl esters 1-8c and this has indicated a strong preference for phenylalanine but flexibility in the N-protection that may be used. Furthermore, we have successfully exploited striking differences in the rate of reaction of carbohydrate acyl acceptors in this system to perform exclusively mannose over N-acetylglucosamine selective one-pot acylations. We have recently reported greatly broadened substrate amino acid ester specificities for glycosylated variants of SBL (Matsumoto et al., (2001)) and we are currently exploring transesterifications catalyzed by these glyco-SBLs with 4-8c and other donors the results of which will be reported in due course.

We thank the BBSRC for generous funding, Genencor International for SBL, and Altus for TL-CLEC. We thank the EPSRC for access to the Mass Spectrometry Service at Swansea and the Chemical Database Service at Daresbury.

Scheme 1 Reagents and Conditions: i, For 1b AC₂₀, MeOH; 2b Boc₂O, NaOH (aq); 3b ZCl, toluene, NaOH (aq); 4b p-TsOH, BnOH, benzene, reflux then HBr/AcOH then FmocCl, Na₂CO₃, dioane:H₂O (3:5); 5b p-TsOH, BnOH, benzene, reflux then CuSO₄, EtOH, NaOH (aq), pH 8 then FmocCl, Na₂CO₃, dioane:H₂O (3:5); 6b p-TsOH, BnOH, benzene, reflux-then HBr/AcOH then Boc₂O, Na₂CO₃ i-PrOH:H₂O (2:1), 0° C.; 7b p-TsOH, BnOH, benzene, reflux then HI/AcOH, 50° C. then (Boc)₂O, Na₂CO₃ i-PrOH:H₂O (2:1), 0° C.; 8b ZCl, toluene, NaOH (aq) then p-TsOH, BnOH, benzene, reflux then NaOH (aq), dioane:H₂O (5:1); ii, Pd(OAc)₂, vinyl acetate, KOH, yield for a→c 1c 40%; 2c 68%; 3c 60%; 4c 80%; 5c 51%; 6c 72%; 7c 65%; 8c, 16%.

Supplementary Information

Vinyl N-acetyl-L-phenylalaninate 1c

A mixture of N-acetyl-L-phenylalanine 1b (10 g, 48 mmol), vinyl acetate (450 mL, 4.8 mol), palladium acetate (2.1 g, 9.3 mmol) and potassium hydroxide (270 mg, 4.8 mmol) was stirred for 24 h at r.t. The mixture was then poured into ether (1.5 L) and filtered through a celite bed. After evaporation in vacuo the crude product was purified by flash chromatography (hexane: EtOAc 1:1 v/v) to give vinyl N-acetyl-L-phenylalaninate 1c (4.5 g, 40%); m.p. 90-93° C.; [α]_{D}²⁵+29.6 (c 0.4, CHCl₃); [lit. m.p. 90.0-91.0° C.; [α]_{D}²⁵+32.7 (c 1.05, CHCl₃)]; ¹³C NMR (50 MHz, CDCl₃) β168.8, 167.9 (C═O), 139.7 (CH vinyl), 134.4, 128.3, 127.7, 126.2 (C arom.), 98.2 (CH₂ vinyl), 51.9 (Cα), 36.5 (Cp), 22.0 (NHCOCH₃); ¹H NMR (300 MHz, CDCl₃) β 7.3-7.0 (6H, m, Ar, CH₂═CH—), 5.90 (sbr, 1H, NH), 4.81 (2H, m), 4.63 (dd, J 1.8 Hz, J 6.3 Hz, 1H), 3.08 (2H, m), 1.9 (s, 3H, NHCOCH₃); m/z (ES⁺): 256 (100, [M+Na]⁺).

N-tert-Butyloxycarbonyl-L-phenylalanine Vinyl Ester 2c

L-phenylalanine (5 g; 0.03 mol) was dissolved in an aqueous solution of sodium hydroxide (1N; 60 mL) and di-tert-butyl-dicarbonate (8g; 0.04 mol) in solution in dioxane (20 mL) was slowly added at 0° C. After one night, the mixture was neutralised with an aqueous solution of HCl (1N), and extracted with ethyl acetate (3 times). The organic layers were dried over sulfate magnesium and concentrated in vacuo to give a white solid 2b. The crude acid 2b was dissolved in vinyl acetate (280 mL; 100 eq.), then palladium acetate (1.3 g; 0.2 eq.) and potassium hydroxide (168 mg; 0.1 eq.) were added. The mixture was stirred overnight at r.t., then poured into diethyl ether and filtered through a celite bed. After concentration in vacuo, the crude product was purified by flash chromatography (hexane/ethyl acetate 6/1 v/v) to give the acyl donor 2c as a pale yellow oil (6 g; 68% over two steps): [α]_{D}²⁵+9.3 (c 1.82, CHCl₃); Vma. (film): 3436 cm⁻¹ (NH), 1757 cm⁻¹ (C═O), 1712 cm⁻¹ (C═O), 1647 cm⁻¹, 1497 cm⁻¹; ¹³C NMR (125 MHz, CDCl₃) β 169.4, 155.3 (2×C═O), 141.1 (CH vinyl), 135.8, 129.5, 128.9, 127.4 (C arom), 99.2 (CH₂ vinyl), 80.4 (C(CH₃)₃), 54.5 (Cα), 38.2 (Cβ), 28.5 (C(CH₃)₃); ¹H NMR (500 MHz, CDCl₃) β 7.31-7.14 (m, 6H, Ar, CH₂═CH—), 4.98 (d br, J 8.7 Hz; 1H; NH, 4.94 (dd, J 1.9 Hz, J 13.8 Hz, 1H, CHH′═CH—), 4.68 (m, 1H, CHH′═CH—), 4.66 (dd, J 1.7 Hz; J 6.2 Hz, 1H, Hα), 3.17 (dd, J 6.2 Hz, J 14.5 Hz, 1H, CHH′), 3.11 (dd, J 1.7 Hz, J 14.5 Hz, 1H, CHH′); MS (ES⁺) m/z=292 (20, [M+H]⁺), 314 (40, [M+Na]⁺), 330 (100, [M+K]⁺); HRMS (ES⁺): C₁₆H₂₂O₄N calculated 292.1549; measured 292.1547 [M+H]⁺.

N-Benzyloxycarbonyl-L-phenylalanine Vinyl Ester 3c

L-phenylalanine (0.5 g; 3.0 mmol) was dissolved in a mixture of toluene (6 mL) and aqueous solution of sodium hydroxide (3N; 4.5 mL). Then, benzyl chloroformate (0.6 mL; 1.3 eq.) was added at 0° C. After 16 h, the layers were separated and the organic layer was washed with an aqueous solution of sodium hydroxide (3N). The combined aqueous layers were acidified with an aqueous solution of HCl (3N), then extracted with chloroform. The combined organic layers were dried over magnesium sulfate and concentrated in vacuo to give a white solid. The crude acid was dissolved in vinyl acetate (30 mL; 100 eq.), and palladium acetate (136 mg; 0.2 eq.) and potassium hydroxide (17 mg; 0.1 eq.) were added. The mixture was stirred overnight at r.t., then poured into diethyl ether and filtered through a celite bed. After concentration in vacuo, the crude product was purified by flash chromatography (hexane/ethyl acetate 5/1 v/v) to give the vinyl ester 3c as a colourless oil (0.6 g; 60% over two steps): [α]_{D}²⁵+17.9 (c 0.57, CHCl₃); νₘₐₓ (film): 3332 cm⁻¹ (NH), 1758 cm⁻¹ (C═O), 1721 cm⁻¹ (C═O), 1646 cm⁻¹, 1511 cm⁻¹; ¹³C NMR (125 MHz, CDCl₃) δ 168.8, 155.6 (2×CO); 140.8 (CH vinyl), 136.1, 135.2, 129.3, 128.7, 128.5, 128.2, 128.1, 127.3 (C arom), 99.2 (CH₂ vinyl), 67.1 CH₂ benzyl), 54.6 (Cα), 37.9 (Cβ); ¹H NMR (500 MHz, CDCl₃) δ 7.40-7.10 (11H; m; Ar, CH₂═CH—), 5.24 (d, J 8.9 Hz, 1H, NH), 5.12 (s; 2H; CH₂ benzyl group), 4.96 (dd; J 1.8 Hz; J 14.9 Hz; 1H; CHH′═CH—); 4.76 (dd; J5.9 Hz; J 14.9 Hz; 1H; CHH′═CH—); 4.68 (dd; J 1.7 Hz; J 6.1 Hz; 1H; Hα); 3.20 (dd; J 6.2 Hz; J 10.9 Hz; 1H; CHH′), 3.15 (dd; J 1.7 Hz; J 10.9 Hz; 1H; CHH′); MS (ES⁺) m/z=326 (55, [M+H]⁺); 348 (48, [M+Na]⁺); 364 (100, [M+K]⁺); HRMS (ES⁺): C₁₉H₂₀O₄N calculated 326.1392; measured 326.1391 [M+H]⁺.

Benzyl β-vinyl-N-(9-fluorenylmethoxycarbolnyl)-L-aspartate 4c

Benzyl N-(9-fluorenylmethoxycarbonyl)-L-aspartate 4bⁱ (0.5 g; 1.12 mmol) was dissolved in vinyl acetate (100 eq.; 10.5 in L). Palladium acetate (0.2 eq.; 50 mg) and potassium hydroxide (0.1 eq.; 6 mg) were added. The mixture was stirred for 24 h at r.t. The reaction mixture was poured into diethyl ether and filtered through a celite bed. After evaporation in vacuo, the crude product was purified by flash chromatography (hexane:ethyl acetate 5:1 v/v) to give vinyl ester 4c (421 mg; 80%): mp 93-95° C. (hexane:ethyl acetate); [α]_{D}²⁵+6.4 (c, 0.36 CHCl₃); νₘₐₓ (film): 3432 cm⁻¹ (NH), 1754 cm⁻¹ (C═O), 1646 cm⁻¹ (amide I), 1510 cm⁻¹ (amide II); ¹H NMR (500 MHz; CDCl₃) β: 7.77-7.59, 7.43-7.30 (m; 13H; benizyl and Fmoc), 7.00 (dd, J 6.0 Hz, J 13.7 Hz, 1H, CH₂═CH—), 5.83 (d, J 8.5 Hz, 1H, NH), 5.23 (s, CH₂ benzyl group), 4.92 (dd, J 1.5 Hz, J 13.7 Hz, 1H, CHH′═CH—), 4.73 (m, 1H, CHH′═CH—), 4.64 (dd, J 1.5 Hz, J 6.0 Hz, 1H), 4.39 (m, 2H, CH₂ Fmoc), 4.23 (pt, J 7.0 Hz, 1H, CH Fmoc), 3.16 (dd; J4.5 Hz, J 17.5 Hz, 1H, CHH′); 2.99 (dd; J4.5 Hz; J 17.5 Hz, CHH′); ¹³C NMR (125 MHz; CDCl₃) β: 170.5, 168.3, 156.1 (3×C═O); 144.0, 143.9, 141.5, 141.5, 140.9, 135.2, 128.9, 128.6, 127.9, 127.3, 125.4, 120.2 (C arom, CH vinyl); 98.9 (CH₂ vinyl); 68.0, 67.6 (CH₂ benzyl, CH₂ Fmoc); 50.6, 47.3 (2×CH); 36.7 (CH₂); MS (ES⁺) m/z=494 (100, [M+Na]⁺); HRMS (ES⁺): calculated for C₂₈H₂₆NO₆ 472.1760; measured 472.1769 [M+H]⁺.

Vinyl β-benzyl-N-(9-fluorenylmethoxycarbonil)-L-aspartate 5c

β-Benzyl-N-(9-fluorenylmethoxycarbonyl)-L-aspartic acid 5b (0.5 g; 1.12 mmol) was dissolved in vinyl acetate (100 eq.; 10.5 mL). Palladium acetate (0.2 eq.; 50 mg) and potassium hydroxide (0.1 eq.; 6 mg) were added. The mixture was stirred for 24 h at r.t. The mixture was poured into diethyl ether and filtered through a celite bed. After evaporation in vacuo, the crude product was purified by flash chromatography (hexane:ethyl acetate 5:1 v/v) to give vinyl ester 5c (287 mg; 51%): mp 53-55° C. (hexane:ethyl acetate); [α]_{D}²⁵+8.4 (c, 0.57 CHCl₃); νₘₐₓ (film): 3427 cm⁻¹ (NH), 1759 cm⁻¹ (C═O), 1729 cm⁻¹ (C═O), 1647 cm⁻¹ (amide I), 1508 cm⁻¹ (amide II); ¹H NMR (500 MHz; CDCl₃), β: 7.78-7.61, 7.43-7.30 (m, 13H; benzyl and Fmoc Ar); 7.25 (dd; J6.0 Hz; J 13.2 Hz; 1H, CH₂═CH—); 5.83 (d; J8.5 Hz; 1H; NH); 5.17 (s; 2H; CH₂ benzyl); 4.93 (d; J 13.2 Hz; 1H, CHH′═CH—); 4.77 (m; 1H; CHH′═CH—); 4.59 (m; 1H); 4.37 (m; 2H, CH₂ Fmoc); 4.25 (pt; J 7.0 Hz; 1H, CH Fmoc); 3.16 (dd; J4.5 Hz; J 17.2 Hz; 1H, CHH′); 2.97 (dd; J4.5 Hz; J 17.0 Hz; 1H, CHH′); ¹³C NMR (125 MHz; CDCl₃) β: 170.8, 168.3, 156.1 (3×C═O); 144.0, 143.9, 141.5, 141.5, 141.3, 135.4, 128.9, 128.6, 128.0, 127.3, 125.3, 120.3 (C arom, CH vinyl); 99.5 (CH₂ vinyl); 67.6, 67.3 (CH₂ benzyl and CH₂ Fmoc); 50.4, 47.3 (2×CH); 36.8. (CH₂); MS (ES⁺): m/z494 (100, [M+Na]⁺); HRMS (ES⁺): calculated for C₂₈H₂₆NO₆ 472.1760; measured 472.1766 [M+H].

Benzyl β-vinyl-N-(t-butyloxycarbonyl)-L-aspartate 6c

Benzyl N-(t-butylcarbonyl)-L-aspartate 6b (0.2 g; 0.62 mmol) was dissolved in vinyl acetate (100 eq.; 5.6 mL). Palladium acetate (0.2 eq.; 28 mg) and potassium hydroxide (0.1 eq.; 3.5 mg) were added. The mixture was stirred for 24 h at room temperature. The mixture was poured into diethyl ether and filtered through a celite bed. After evaporation in vacuo, the crude product was purified by flash chromatography (hexane:ethyl acetate 9:1 then 7/3 v/v) to give vinyl ester 6c (156 mg; 72%) as an oil: [β]_{D}²⁵+15.1 (c, 0.6 CHCl₃); νₘₐₓ (film): 3440 cm⁻¹ (NH), 1751 cm⁻¹ (C═O), 1699 cm⁻¹ (C═O), 1648 cm⁻¹ (amide I), 1498 cm⁻¹ (amide II); ¹H NMR (500 MHz; CDCl₃) β: 7.32-7.29 (m; 5H arom; benzyl); 7.17 (dd; J7.2 Hz; J 14.8 Hz; CHH′═CH—); 5.47 (d; J9.6 Hz; 1H; NH); 5.17 (s; 2H; CH₂ benzyl); 4.86 (dd; J 1.9 Hz; J 14.8 Hz; 1H, CHH′═CH—); 4.60 (m; 1H, Hα); 4.58 (dd; J 1.9 Hz; J 7.2 Hz; CHH′═CH—); 3.08 (dd; J 3.7 Hz; J 17.6 Hz; 1H, CHH′); 2.91 (dd; J 4.9 Hz; J 16.8 Hz; 1H, CHH′); 1.41 (s; 9H; C(CH₃)₃); ¹³C NMR (125 MHz; CDCl₃) β: 170.9, 168.4, 155.6 (3×CO); 140.9 (CH vinyl), 135.4, 128.8, 128.5, 127.8 (C arom); 98.8 (CH₂ vinyl); 80.5 (C(CH₃)₃); 67.8 (CH₂ benzyl); 50.1 (CH); 36.8 (CH₂); 28.5 (C(CH₃)₃); MS: m/z=372 (100, [M+Na]⁺); HRMS (ES⁺): C₁₈H₂₇N₂O₆ calculated 367.1869; measured 367.1867 [M+NH₄]+.

Benzyl β-vinyl-N-(t-butyloxycarbonyl)-L-glutamate 7c

Benzyl N-(t-butylcarbonyl)-L-glutamate 7b (0.3 g; 0.89 mmol) was dissolved in vinyl acetate (100 eq.; 8.2 mL). Palladium acetate (0.2 eq.; 40 mg) and potassium hydroxide (0.1 eq.; 5.0 mg) were added. The mixture was stirred for 24 h at r.t. The mixture was poured into diethyl ether and filtered through a celite bed. After evaporation in vacuo, the crude product was purified by flash chromatography (hexane: ethyl acetate 9:1 then 7/3 v/v) to give vinyl ester 7c (210 mg; 65%): mp 43-45° C. (hexane:ethyl acetate); [α]_{D}²⁵+4.8 (c, 0.3 CHCl₃); νₘₐₓ (film): 3433 cm⁻¹ (NH), 1747 cm⁻¹ (C═O), 1720 cm⁻¹ (C═O), 1647 cm⁻¹ (amide I), 1500 cm⁻¹ (amide II); ¹H NMR (500 MHz; CDCl₃) β: 7.38-7.28 (m; 5H arom; benzyl); 7.26 (dd; J 6.4 Hz; J 13.9 Hz; 1H, CH₂═CH—); 5.21 (d; J9.0 Hz; 1H; NH); 5.18 (s; 2H; CH₂ benzyl); 4.88 (dd; J 1.7 Hz; J 13.9 Hz; 1H, CHH′═CH—); 4.58 (dd; J 1.7 Hz; J 6.4 Hz; 1H CHH′═CH—); 4.41 (m; 1H); 2.47 (m; 2H); 2.24 (m; 1H); 10.99 (m; 1H); 1.43 (s; 9H; C(CH₃)₃); ¹³C NMR (125 MHz; CDCl₃) β: 171.9, 169.8, 155.3 (3×CO); 141.0., 135.1, 128.6, 128.5, 128.3 (C arom and CH vinyl); 97.9 (CH₂ vinyl); 80.1 (C(CH₃)₃); 67.3 (CH₂ benzyl); 52.8 (CH); 29.9 (CH₂); 28.2 (C(CH₃)₃); 27.4 (CH₂); MS (ES⁺): m/z=386 (100, [M+Na]⁺); HRMS (ES+): C₁₉H₂₆NO₆ calculated 364.1760; measured 364.1757 [M+H]⁺.

Vinyl β-benzyl-N-(benzyloxycarbonyl)-L-glutamate 8c

β-Benzyl-N-(benzyloxycarbonyl)-L-glutamic acid 8b (0.3 g; 0.61 mmol) was dissolved in vinyl acetate (100 eq.; 7.4 mL). Palladium acetate (0.2 eq.; 36 mg) and potassium hydroxide (0.1 eq.; 4.5 mg) were added. The mixture was stirred for 24 h at r.t. The mixture was poured into diethyl ether and filtered through a celite bed. After evaporation in vacuo, the crude product was purified by flash chromatography (hexane:ethyl acetate 9:1 then 7/3 v/v) to give the acyl donor 8c (51 mg; 16%) as an oil: [α]_{D}²⁵+10.0 (c, 0.2 CHCl₃); νₘₐₓ (film): 3433 cm⁻¹ (NH), 1726 cm⁻¹ (C═O), 1660 cm⁻¹ (amide I), 1507 cm⁻¹ (amide II); ¹H NMR (500 MHz; CDCl₃) β: 7.37-7.33 (m; 10H arom; benzyl); 7.21 (dd; J 6.3 Hz; J 14.2 Hz; CHH′═CH—); 5.42 (d; J 6.5 Hz; 1H; NH); 5.08 (s; 4H; 2×CH₂ benzyl); 4.93 (dd; J 1.4 Hz; J 14.2 Hz; CHH′═CH—); 4.64 (dd; J 1.4 Hz; J 6.3 Hz; 1H CHH′═CH—); 4.48 (m; 1H); 2.48 (m; 2H); 2.28 (m; 1H); 2.03 (m; 1H); ¹³C NMR (125 MHz; CDCl₃) β: 172.4, 169.1, 155.9 (3×CO); 140.8, 136.0, 135.6, 128.5, 128.2, 128.1 (C arom, CH vinyl); 99.2 (CH₂ vinyl); 67.1, 66.6 (2×CH₂ benzyl); 53.2 (CH); 30.1, 27.2 (2×CH₂); MS (ES⁺): m/z=420 (100, [M+Na]⁺); HRMS (ES⁺): C₂₂H₂₄NO₆ calculated 398.1604; measured 398.1601 [M+H]⁺.

Preparation of Subtilisin Bacillus lentus (SBL)

50 mg of pure lyophilised SBL was added to 5 mL of 0.1M phosphate buffer (H 8.0) and freeze-dried.

General Procedure for SBL-Catalyzed Acylation

0.56 mmol of carbohydrate acyl-acceptor 9-20a, 0.89 mmol (1.6 equiv.) amino acid vinyl ester 1-8c (1.6 eq.) and 10 mg of pH adjusted SBL preparation were suspended in 5 mL of anhydrous pyridine and stirred under nitrogen at 45° C. for 120 h. In all cases, no background reaction in the absence of SBL was detected. The reaction mixture was filtered through celite, evaporated and the residue purified by flash chromatography (MeOH:EtOAc, 1:19 or CHCl₃:MeOH:AcOH:H₂O, 90:10:0.5:1 or CHCl₃:MeOH:AcOH:H₂O, 85:13:0.5:1.5) to give the following acylated sugars 9-20b,c:

6-O-carboxy-(N-acetyl-phenylalanine)-α,β-D-glucopyranose 9b: [α]_{D}²⁵+31.6 (c, 0.50 in MeOH); νₘₐₓ (KBr): 3424 cm⁻¹ (OH, NH), 1758 cm⁻¹ (C═O), 1652 cm⁻¹ (amide I), 1540 cm⁻¹ (amide II); ¹H NMR (500 MHz; CD₃OD, α,β=56:44): δ 7.28-7.24 (m; 5H; H arom); 5.12 (d; J 3.9 Hz; 1H; H-1α); 4.71 (dd; J 5.6 Hz; J 7.9 Hz; 1H; Hα); 4.51 (d; J7.1 Hz; 1H; H-1β); 4.44 (m; 1H; H-6); 4.26 (m; 1H; H-6′); 4.01 (m; 1H); 3.70 (t; J 6.6 Hz; 1H); 3.38 (m; 1H); 3.21 (m; 1H; CHH′β); 3.17 (m; 1H); 2.96 (m; 1H; CHH′β); 1.91 (s; 3H; NHCOCH₃); ¹³C NMR (125.7 MHz; CD₃OD): δ 172.1, 171.8, 171.7 (CO); 137.1, 137.0, 129.1, 129.08, 129.0, 128.3, 126.7 (C arom); 97.0 (C-1β); 92.8 (C-1α); 76.7, 75.0, 74.0, 73.6, 72.6, 70.8, 70.5, 69.4 (C-2; C-3; C-4; C-5); 64.6, 64.5 (C-6); 54.2, 54.1 (CH amino acid); 37.2, 37.1 (CH₂ amino acid); 21.2, 21.1 (NHCOCH₃); MS (ES⁺): m/z=392 (100, [M+Na]⁺); HRMS (ES⁺): calculated 392.1321; measured 392.1315 [M+Na]⁺.

6-O-carboxy-(N-acetyl-phenylalanine)-α,β-D-galactopyranose 10b: [α]_{D}²⁵+37.6 (c, 0.69 in MeOH); νₘₐₓ (KBr): 3436 cm⁻¹ (OH, NH), 1758 cm⁻¹ (C═O), 1656 cm⁻¹ (amide I), 1540 cm⁻¹ (amide II); ¹H NMR (500 MHz; CD₃OD, α,β=1:1): β7.30-7.21 (m; 5H; H arom); 5.11 (d; J4.6 Hz; H-1α); 4.72 (dd; J5.0 Hz; J9.3 Hz; 1H; Hα); 4.51 (d; J 7.8 Hz; H-1β); 4.44 (m; 1H; H-6); 4.26 (m; 1H; H-6′); 4.01 (m; 1H); 3.70 (m; 1H); 3.39 (m; 1H); 3.20 (m; 1H); 3.15 (m; 1H; CHH′β); 2.96 (m; 1H; CHH′β); 1.91 (s; 3H; NHCOCH₃); ¹³C NMR (125.7 MHz; CD₃OD): β172.1, 171.8, 171.7 (CO); 137.1, 129.1, 129.0, 128.3, 126.7 (C arom); 97.0 (C-1β); 92.8 (C-1α); 76.7, 74.9, 74.0, 73.6, 72.5, 70.8, 70.5, 69.4 (C-2; C-3; C-4; C-5); 64.6, 64.5 (C-6); 54.2, 54.1 (CH amino acid); 37.2, 37.1 (CH₂ amino acid); 21.2, 21.1 (NHCOCH₃); MS (ES⁺): m/z=392 (100, [M+Na]⁺); HRMS (ES⁺): calculated 392.1321; measured 392.1321 [M+Na]⁺.

6-O-carboxy-(N-acetyl-phenylalanine)-α-D-mannopyranose 11b: [α]_{D}²⁵+10.8 (eqlbm) (c, 0.81 in MeOH); νₘₐₓ (KBr): 3280 cm⁻¹ (OH, NH), 1744 cm⁻¹ (C═O), 1648 cm⁻¹ (amide I), 1552 cm⁻¹ (amide II); ¹H NMR (500 MHz; CD₃OD): β7.27-7.20 (m; 5H; H arom); 5.09 (s; 1H; H-1α); 4.75 (dd; J 5.4 Hz; J9.4 Hz; 1H; Ha); 4.44 (d; J 11.7 Hz; 1H; H-6); 4.31 (dd; J 7.4 Hz; J 11.7 Hz; 1H; H-6′); 3.97 (dd; J 7.4 Hz; J 8.9 Hz; 1H; H-5); 3.81 (m; 2H; H-2, H-3); 3.66 (t; J8.9 Hz; 1H; H-4); 3.24 (dd; J5.4 Hz; J 13.3 Hz; 1H; CHH′β; 2.96 (dd; J9.4 Hz; J 13.3 Hz; 1H; CHH′β); 1.91 (s; 3H; NHCOCH₃); ¹³C NMR (125.7 MHz; CD₃OD): β172.2, 171.8 (2×CO); 137.1, 129.1, 129.0, 128.3, 126.7 (C arom); 94.8 (C-1); 71.7, 71.0, 70.4, 67.6 (C-2; C-3; C-4; C-5); 64.7 (C-6); 54.1 (CH amino acid); 37.1 (CH₂ amino acid); 21.2 (NHCOCH₃); MS (ES⁺): m/z=392 (100, [M+Na]⁺); HRMS (ES⁺): calculated 392.1321; measured 392.1321 [M+Na]⁺.

Methyl 6-O-carboxy-(N-acetyl-L-phenylalanine)-α-D-glucopyranoside 13b: [α]_{D}²⁵+82.6 (c, 0.53 in MeOH); νₘₐₓ (KBr): 3416 cm⁻¹ (OH, NH), 1748 cm⁻¹ (C═O), 1658 cm⁻¹ (amide I), 1546 cm⁻¹ (amide II); ¹H NMR (500 MHz; CD₃OD): β7.31-7.19 (m; 5H arom); 4.72 (dd; J 4.9 Hz; J 8.5 Hz; 1H; Ha); 4.66 (d; J 4.1 Hz; 1H; H-1); 4.42 (dd; J2.5 Hz; J 11.7 Hz; 1H; H-6); 4.25 (dd; J6.4; J 11.7 Hz; 1H; H-6′); 3.75 (ddd; J2.5 Hz; J6.4 Hz; J 10.1 Hz; H-5); 3.62 (t; J9.5 Hz; H-3); 3.40-3.38 (m and s; 4H; H-4 and OCH3); 3.26 (t; J 9.0 Hz; 1H; H-2); 3.21 (dd; J 4.9 Hz; J 14.3 Hz; 1H; CHH′β); 2.96 (dd; J 8.5 Hz; J 14.3 Hz; 1H; CHH′β); 1.90 (s; 3H; NHCOCH₃); ¹³C NMR (125.7 MHz; CD₃OD): β172.0, 171.7 (2×CO); 137.1, 129.0, 128.3, 126.7 (C arom); 100.1 (C-1); 73.8 (C-3); 72.2 (C-4); 70.7 (C-2); 69.5 (C-5); 64.6 (C-6); 54.5 (OCH₃); 54.1 (CH amino acid); 37.1 (CH₂ amino acid); 21.1 (NHCOCH₃); MS (ES⁺): m/z406 (100, [M+Na]⁺); HRMS (ES⁺): calculated 384.1658; measured 384.1662 [M+H1].

Methyl 6-O-carboxy-(N-acetyl-L-phenylalanine)-β-D-glucopyranoside 14b: [α]_{D}²⁵−17.9 (c, 0.56 in MeOH); νₘₐₓ (KBr): 3422 cm⁻¹ (OH, NH), 1753 cm⁻¹ (C═O), 1652 cm⁻¹ (amide I), 1544 cm⁻¹ (amide II); ¹H NMR (500 MHz; CD₃OD): β7.33-7.17 (m; 5H arom); 4.73 (dd; J4.9 Hz; J8.1 Hz; 1H; Hα); 4.44 (dd; J2.6 Hz; J 11.8 Hz; 1H; H-6); 4.28 (dd; J6.0 Hz; J 11.8 Hz; 1H; H-6′); 4.20 (d; J8.0 Hz; 1H; H-1); 3.54-3.47 (m and s; 4H; H-5 and OCH₃); 3.29 (t; J 8.9 Hz; 11H; H-4); 3.21 (dd; J4.9 Hz; J 14.0 Hz; 1H; CHH′β); 3.18 (dd; J9.2 Hz; J8.0 Hz; 1H; H-2); 2.96 (dd; J8.1 Hz; J 14.0 Hz; 1H; CHH′β); 1.91 (s; 3H; NHCOCH₃); ¹³C NMR (125.7 MHz; CD₃OD): β173.2, 172.8 (2×CO); 138.2, 130.2, 129.5, 127.9 (C arom); 105.4 (C-1); 77.8 (C-3); 75.1, 74.9 (C4, C-5); 71.6 (C-2); 65.5 (C-6); 57.3 (OCH₃); 55.3 (CH amino acid); 38.3 (CH₂ amino acid); 22.2 (NHCOCH₃); MS (ES⁺): m/z406 (100, [M+Na]⁺); HRMS (ES⁺): calculated 406.1478; measured 406.1475 [M+Na];

Methyl 6-O-carboxy-(N-acetyl-L-phenylalamnie)-β-D-galactopyranoside 15b: [α]_{D}²⁵−1.9 (c, 0.70 in MeOH);); [α]_{D}²⁵+15.6 (c 2.5, H₂O) for a 95% mixture of 15b with other products]; νₘₐₓ (KBr): 3428 cm⁻¹ (OH, NH), 1758 cm⁻¹ (C═O), 1656 cm⁻¹ (amide I), 1540 cm⁻¹ (amide II); ¹H NMR (500 MHz; CD₃OD): β7.28-7.21 (m; 5H; H arom); 4.68 (dd; J 6.0 Hz; J 8.7 Hz; 11H; Hα); 4.34 (dd; J 8.0 Hz; J 11.0 Hz; 11H; H-6); 4.23 (dd; J 5.3 Hz; J 11.0 Hz; 1H; H-6′); 4.13 (d; J 7.6 Hz; 1H; H-1); 3.71 (dd; J 1.4 Hz; J 3.3 Hz; 1H; H-4); 3.61 (ddd; J 1.4 Hz; J 5.3 Hz; J 8.0 Hz; 1H; H-5); 3.50 (s; 3H; OCH₃); 3.49 (dd; J 3.3 Hz; J 10.2 Hz; H-3); 3.45 (dd; J 7.6 Hz; J 10.2 Hz; 1H; H-2); 3.15 (dd; J 6.0 Hz; J 13.6 Hz; 1H; CHH′β); 2.99 (dd; J 8.7 Hz; J 13.6 Hz; 1H; CHH′β); 1.92 (s; 3H; NHCOCH₃); ¹³C NMR (125.7 MHz; CD₃OD): β172.1, 171.7 (2×CO); 137.3, 129.0, 128.4, 126.8 (C arom); 104.7 (C-1); 73.5, 72.5, 71.1, 68.9 (C-2; C-3; C-4; C-5); 63.9 (C-6); 56.1 (OCH₃); 54.2 (CH amino acid); 37.2 (CH₂ amino acid); 21.1 (NHCOCH₃); MS (ES⁺): m/z=406 (100, [M+Na]⁺); HRMS (ES⁺): calculated 406.1478; measured 406.1474 [M+Na];

Methyl 6-O-carboxy-(N-acetyl-L-phenylalanine)-α-D-mannopyranoside 16b: [α]_{D}²⁵+35.0 (c, 0.62 in MeOH); νₘₐₓ (KBr): 3404 cm⁻¹ (OH, NH), 1751 cm⁻¹ (C═O), 1654 cm⁻¹ (amide I), 1538 cm⁻¹ (amide II); ¹H NMR (500 MHz; CD₃OD): β7.28-7.22 (m; 5H; H arom); 4.75 (dd; J 5.7 Hz; J 9.5 Hz; 1H; Ha); 4.63 (d; J 1.6 Hz; H-1); 4.44 (dd; J 1.8 Hz; J 11.6 Hz; 1H; H-6); 4.30 (dd; J 6.2 Hz; J 11.6 Hz; 1H; H-6′); 3.70 (m; 1H; H-5); 3.89 (m; 1H; H-3); 3.80 (dd; J 1.6 Hz; J 3.2 Hz; 1H; H-2); 3.65 (t; J 9.6 Hz; H-4); 3.37 (s; 3H; OCH₃); 3.22 (dd; J 5.7 Hz; J 13.9 Hz; 1H; CHH′β); 2.96 (dd; J 9.5 Hz; J 13.9 Hz; 1H; CHH′β); 1.90 (s; 3H; NHCOCH₃); ¹³C NMR (125.7 MHz; CD₃OD): β171.7, 172.0 (2×CO); 137.1, 129.1, 128.3, 126.7 (C arom); 101.6 (C-1); 71.3, 70.8, 70.7, 67.4 (C-2; C-3; C-4; C-5); 64.7 (C-6); 54.2 (CH amino acid), 54.1 (OCH₃); 37.2 (CH₂ amino acid); 21.1 (NHCOCH₃); MS (ES⁺): m/z=406 (100, [M+Na]⁺); HRMS (ES⁺): calculated 401.1924; measured 401.1916 [M+NH₄]⁺.

Phenyl 6-O-carboxy-(N-acetyl-L-phenylalanine)-1-thio-β-D-glucopyranoside 17b: [α]_{D}²⁵−17.0 (c, 0.43 MeOH); νₘₐₓ (KBr): 3412 cm⁻¹ (OH, NM, 1740 cm⁻¹ (C═O), 1658 cm⁻¹ (amide I), 1538 cm⁻¹ (amide II); ¹H NMR (500 MHz; CD₃OD): δ 7.53 (dd; J 8.2 Hz; J 1.5 Hz; 2H; H arom); 7.28-7.18 (m; 8H; H arom); 4.73 (dd; J 5.3 Hz; J 8.8 Hz; 1H; Ha); 4.67 (d; J9.5 Hz; 1H; H-1); 4.47 (dd; J 1.7 Hz; J 11.6 Hz; 1H; H-6); 4.21 (dd; J 6.0 Hz; J 11.6 Hz; 1H; H-6′); 3.55 (ddd; J 1.7 Hz; J 6.0 Hz; J 9.5 Hz; 1H; H-5); 3.40 (t; J 9.4 Hz; 1H; H-3); 3.25 (pt, J 9.0 Hz, 1H, H-4), 3.22 (dd, J 8.5 Hz, J9.5 Hz, 1H, H-2); 3.14 (dd; J4.7 Hz; J 13.8 Hz; 1H; CHH′β); 2.91 (dd; J 8.8 Hz; J 13.2 Hz; 1H; CHH′β); 1.91 (s; 3H; NHCOCH₃); ¹³C NMR (125.7 MHz; CD₃OD): β173.2, 172.8 (2×CO); 138.1, 134.8, 132.9, 130.3, 129.9, 129.5, 128.4, 127.8 (C arom); 88.9 (C-1); 79.4 (C-3); 78.9 (C-4); 73.6, 71.5 (C-2, C-5); 65.8 (C-6); 55.2 (CH amino acid); 38.3 (CH₂ amino acid); 22.3 (NHCOCH₃); MS (ES⁺): m/z=484 (100, [M+Na]⁺); HRMS (ES⁺): calculated 462.1586; measured 462.1594 [M+H].

Phenyl 3-O-carboxy-(N-acetyl-L-phenylalanine)-1-thio-β-D-glucopyranoside 17c: [α]_{D}²⁵+4.0 (c, 0.1 CHCl₃); νₘₐₓ (KBr): 3404 cm⁻¹ (OH, NH), 1734 cm⁻¹ (C═O), 1653 cm⁻¹ (amide I), 1544 cm⁻¹ (amide II); ¹H NMR (500 MHz; CD₃OD): β 7.60-7.21 (m; 10H; H arom); 5.02 (t; J 9.3 Hz; H-3); 4.70 (d; J 9.5 Hz; 1H; H-1); 4.65 (dd; J 2.0 Hz; J6.0 Hz; 1H; Hα); 3.88 (dd; J2.0 Hz; J 11.6 Hz; 1H; H-6); 3.71 (dd; J4.8 Hz; J 11.6 Hz; 1H; H-6′); 3.53 (t; J 10.0 Hz; 1H; H-2); 3.43-3.39 (m; 2H; H-4 and H-5); 3.00 (dd; J 8.7 Hz; J 13.9 Hz; 1H; CHH′β); 2.90 (dd; J 8.1 Hz; J 13.9 Hz; 1H; CHH′β); 1.91 (s; 3H; NHC(O)CH₃); ¹³C NMR (125.7 MHz; CD₃OD): β171.6, 169.0 (2×CO); 137.3, 137.0, 136.7, 133.8, 129.2, 128.9, 128.3, 126.7 (C arom); 97.9 (C-1); 88.3, 80.6, 70.7, 68.1 (C-2; C-3; C-4; C-5); 61.2 (C-6); 54.2 (CH amino acid); 37.2 (CH₂ amino acid); 21.1 (NHC(O)CH₃); MS (ES⁺): m/z=484 (100, [M+Na]⁺); HRMS (ES⁺): calculated 462.1586; measured 462.1592 [M+H].

Phenyl 6-O-carboxy-(N-acetyl-L-phenylalanine)-1-thio-β-D-galactopyranoside 18b: [α]_{D}²⁵+7.3 (c, 0.2 in MeOH); νₘₐₓ (KBr): 3388 cm⁻¹ (OH, NH), 1743 cm⁻¹ (C═O), 1653 cm⁻¹ (amide I), 1542 cm⁻¹ (amide II); ¹H NMR (500 MHz; CD₃OD): β7.50 (m; 2H; H arom); 7.25-7.16 (m; 8H; H arom); 4.68 (dd; J 5.8 Hz; J 8.7 Hz; 1H; Hα); 4.63 (d; J 9.8 Hz; 1H; H-1); 4.32 (dd; J7.4 Hz; J 11.3 Hz; 1H; H-6); 4.23 (dd; J4.0 Hz; J 11.3 Hz; 1H; H-6′); 3.80 (d; J3.5 Hz; 1H; H-4); 3.70 (ddd; J 1.4 Hz; J4.0 Hz; J7.4 Hz; 1H; H-5); 3.61 (t; J9.4 Hz; 1H; H-2); 3.49 (dd; J3.5 Hz; J9.6 Hz; 1H; H-3); 3.08 (dd; J 3.7 Hz; J 13.4 Hz; 1H; CHH′β); 2.91 (dd; J 8.3 Hz; J 13.4 Hz; 1H; CHH′β); 1.91 (s; 3H; NHCOCH₃); ¹³C NMR (125.7 MHz; CD₃OD): β173.2, 172.8 (2×CO); 130.3-129.5 (C arom); 89.9 (C-1); 77.6, 75.9, 70.8, 70.4 (C-2; C-3; C-4;C-5); 65.9 (C-6); 55.3 (CH amino acid); 38.4 (CH₂ amino acid); 22.2 (NHCOCH₃); MS (ES⁺): m/z=484 (100, [M+Na]⁺); HRMS (ES⁺): calculated 462.1586; measured 462.1587 [M+H]⁺.

Phenyl 6-O-carboxy-(N-acetyl-L-phenylalanine)-1-thio-β-D-mannopyranoside 19b: [α]_{D}²⁵+126.0 (c, 0.25 in MeOH); νₘₐₓ (KBr): 3398 cm⁻¹ (OH, NH), 1750 cm⁻¹ (C═O), 1656 cm⁻¹ (amide I), 1544 cm⁻¹ (amide II); ¹H NMR (500 MHz; CD₃OD): β 7.50 (m; 2H; H arom); 7.40-7.10 (m; 8H; H arom); 5.47 (d; J 1.5 Hz; 1H; H-1); 4.71 (dd; J5.1 Hz; J8.4 Hz; 1H; Hα); 4.64 (dd; J4.6 Hz; J 11.3 Hz; 1H; H-6); 4.32 (dd; J 6.4 Hz; J 11.3 Hz; 1H; H-6′); 4.28 (m, 1H; H-4); 4.12 (dd; J 1.5 Hz; J3.0 Hz; 1H; H-2); 3.73 (m, 1H, H-5), 3.71 (dd; J3.0 Hz; J5.4 Hz; 1H; H-3); 3.12 (dd; J4.5 Hz; J 12.8 Hz; 1H; CHH′β); 2.86 (dd; J 9.0 Hz; J 12.8 Hz; 1H; CHH′β); 1.90 (s; 3H; NHCOCH₃); ¹³C NMR (125.7 MHz; CD₃OD): β171.9, 171.6 (2×CO); 137.4-126.6 (C arom); 89.0 (C-1); 72.3, 71.9, 71.8, 67.7 (C-2; C-3; C-4; C-5); 64.6 (C-6); 53.9 (CH amino acid); 37.1 (CH₂ amino acid); 21.1 (NHCOCH₃); MS (ES⁺): m/z=484 (100, [M+Na]⁺). HRMS (ES⁺): calculated 479.1852; measured 479.1852 [M+NH4]+.

Phenyl 2-N-acetyl-6-O-carboxy-(N-acetyl-L-phenylalanine)-1-seleno-β-D-glucosamine 20b: [α]_{D}²⁵+17.5 (c, 0.1 CHCl₃); νₘₐₓ (KBr): 3288 cm⁻¹ (OH, NH), 1752 cm⁻¹ (C═O), 1653 cm⁻¹ (amide I), 1550 cm⁻¹ (amide II); ¹H NMR (500 MHz; CD₃OD): β 7.57 (m; 2H; H arom); 7.26-7.16 (m; 8H; H arom); 5.01 (d; J 10.0 Hz; 1H; H-1); 4.71 (dd; J4.9 Hz; J9.5 Hz; 1H; Hα); 4.47 (dd; J2.2 Hz; J 11.7 Hz; 1H; H-6); 4.19 (dd; J6.5 Hz; J 11.7 Hz; 1H; H-6′); 3.85 (dd; J 1.7 Hz; J 12.3 Hz; 1H; H-4); 3.83 (t; J 9.3 Hz; 1H; H-2); 3.49 (ddd; J 1.7 Hz; J 6.5 Hz; J 9.5 Hz; 1H; H-5); 3.44 (t; J9.7 Hz; 1H; H-3); 3.12 (dd; J4.9 Hz; J 13.6 Hz; 1H; CHH′β); 2.90 (dd; J 9.5 Hz; J 13.6 Hz; 1H; CHH′β); 1.98 (s; 3H; NHC(O)CH₃); 1.91 (s; 3H; NHC(O)CH₃); ¹³C NMR (125.7 MHz; CD₃OD): β172.4, 172.1, 171.6 (3×CO); 136.9-126.7 (C arom); 83.5 (C-1); 79.0, 75.9, 70.7, 64.6 (C-2; C-3; C-4; C-5); 55.6 (C-6); 54.0 (CH amino acid); 37.1 (CH₂ amino acid); 21.8, 21.1 (2×NHC(O)CH₃); MS (ES⁺): m/z=573 (100, [M+Na]⁺); HRMS (ES⁺): calculated 551.1296; measured 551.1292 [M+H]⁺.

General Procedure for TL-CLEC-Catalyzed Acylation

16a (0.56 mmol), 1c (1.6 eq.) and 3 mg of CLEC-thermolysin (TL-CLEC) were suspended in a mixture of 2.5:0.1 mL of pyridiine: water and stirred under nitrogen at 45° C. for 120 h. The mixture was filtered, concentrated and the residue purified by flash chromatography (CHCl₃:MeOH:AcOH:H₂O, 85:10:0.5:1) to give 16b (48%).

Methyl 6-O-carboxyl-(N-tert-butyloxycarbonyl-L-ohenylalanine)-α-D-mannopyranoside 16c

Methyl α-D-mannopyranoside (50 mg; 0.3 mmol), N-tert-butyloxycarbonyl-L-phenylalanine vinyl ester 2c (119 mg; 1.6 eq.) and 50 mg of pH adjusted SBL preparation were suspended in 5 mL of anhydrous pyridine and stirred under nitrogen at 45° C. for 500 h. The reaction was then concentrated in vacuo and the residue purified by flash chromatography (chloroform/methanol/acetic acid/water 90/4/0.5/1 v/v) to give after lyophilisation the 6-O-acyl sugar 16c as the major compound (79 mg; 63%, (shortened reaction times of 120 h gave 32% of 16c)): [α]_{D}²⁵+37.4 (c 0.64, MeOH); νₘₐₓ (KBr): 3380 cm⁻¹ (OH, NH), 1756 cm⁻¹ (C═O), 1526 cm⁻¹ (amide II), 1457 cm⁻¹; ¹H NMR (500 MHz; CD₃OD): β7.27-7.22 (m, 5H, H arom), 4.63 (d, J 1.5 Hz, 1H, H-1), 4.47 (dd, J 1.9 Hz, J 11.8 Hz, 1H, H-6), 4.44 (m, 1H, Hα), 4.28 (dd; J 6.8 Hz; J 11.8 Hz; 1H; H-6′), 3.81 (dd, J 1.5 Hz, J 3.1 Hz, 1H, H-2), 3.72 (ddd, J 1.9 Hz, J 6.8 Hz, J 9.2 Hz, 1H, H-5), 3.68 (dd, J3.1 Hz, J 8.7 Hz, 1H, H-3), 3.63 (t, J9.4 Hz, 1H, H-4), 3.37 (s, 3H, OCH₃), 3.18 (dd, J4.9 Hz, J 13.8 Hz, 1H, CHH′), 2.92 (dd, J 9.0 Hz, J 13.7 Hz, 1H, CHH′), 1.37 (s, 9H, C(CH₃)₃); ¹³C NMR (125 MHz; CD₃OD): β173.5, 157.7 (2×C═O), 138.4, 130.3, 129.4, 127.7 (C arom), 102.7 (C-1), 80.5 (C(CH₃)₃), 72.5, 71.9, 71.8, 69.7 (C-2, C-3, C-4, C-5), 65.9 (C-6), 56.5 (CH amino acid), 55.4 (OCH₃), 38.7 (CH₂ amino acid), 28.6 (C(CH₃)₃); MS (ES⁺): m/z=464 (100, [M+Na]⁺); HRMS (ES⁺): C₂₁H₃₂O₉N calculated 442.2077; measured 442.2077 [M+H]⁺; and methyl 3-O-carboxyl-(N-tert-butyloxycarbonyl-L-phenylalanine)-α-D-mannopyranoside 16d (20 mg; 17%): [α]_{D}²⁵+31.2 (c 0.12, MeOH); νₘₐₓ (KBr): 3420 cm⁻¹ (OH, NH), 1752 cm⁻¹ (C═O), 1698 cm⁻¹ (C═O), 1526 cml (amide I), 1460 cm⁻¹ (amide II); ¹H NMR (500 MHz; CD₃OD): □□7.33-7.19 (m, 5H, H arom), 5.02 (dd, J3.1 Hz, J9.6 Hz, 1H, H-3), 4.66 (d, J 1.2 Hz, 1H, H-1), 4.44 (dd, J4.4 Hz, J7.3 Hz, 1H, Hca), 3.92 (m, 1H, H-2), 3.90-3.83 (m, 2H, H-6,6′), 3.73 (m, 1H, H-5), 3.61 (m, H-4), 3.41 (s, 3H, OCH₃), 3.25 (dd; J 3.9 Hz, J 14.9 Hz, 1H, CHH′), 2.92 (m, 1H, CH′H), 1.39 (s, 9H, C(CH₃)₃); ¹³C NMR (125 MHz; CD₃OD): β173.2, 158.0 (2×C═O), 138.5, 130.4, 129.4, 127.8 (C arom), 102.5 (C-1), 80.7 (C(CH₃)₃), 76.8 (C-3), 74.7, 74.5, 69.7 (C-2, C-4, C-5), 62.6 (C-6), 56.6 (CH amino acid), 55.3 (OCH₃), 38.6 (CH₂ amino acid), 28.7 (C(CH₃)₃); MS (ES⁺): m/z=464 (100, [M+Na]⁺); HRMS (ES⁺): C₂₁H₃₂O₉N calculated 442.2077; measured 442.2075 [+H]⁺.

Methyl-6-O-carboxyl-(N-benzyloxycarbonyl-L-phenylalanine)-α-D-mannopyranoside 16e

Methyl α-D-mannopyranoside 16a (50 mg; 0.3 mmol), N-benzyloxycarbonyl-L-phenylalanine vinyl ester 3c (134 mg; 1.6 eq.) and 50 mg of pH adjusted SBL preparation were suspended in 5 mL of anhydrous pyridine and stirred under nitrogen at 45° C. for 500 h. The reaction was then concentrated in vacuo and the residue purified by flash chromatography (chloroform/methanol/acetic acid/water 90/4/0.5/1 v/v) to give after lyophilisation the 6-O-acyl sugar 16e (75 mg; 60%): [α]_{D}²⁵+23.2 (c 0.15, MeOH); νₘₐₓ (KBr): 3430 cm⁻¹ (OH, NH), 1740 cm⁻¹ (C═O), 1710 cm⁻¹ (C═O), 1533 cm⁻¹ (amide II), 1451 cm⁻¹; ¹H NMR (500 MHz; CD₃OD): δ 7.32-7.21 (m; 10H; H arom); 5.00 (m; 2H; CH₂ benzyl group); 4.63 (s; 1H; H-1), 4.51 (dd, J5.0 Hz, J9.5 Hz, Ha), 4.47 (dd; J2.2 Hz; J 11.9 Hz; H-6), 4.30 (dd; J6.4 Hz; J 11.9 Hz; 1H; H-6′); 3.80 (dd; J 1.7 Hz; J3.1 Hz; 1H; H-2); 3.70 (m; 1H); 3.65 (m; 2H); 3.30 (s; 3H; OCH₃); 3.23 (dd; J 5.4 Hz; J 12.2 Hz; 1H; CHH′); 2.95 (dd; J 8.8 Hz; J 12.2 Hz; 1H; CHH′); ¹³C NMR (125 MHz; CD₃OD): δ 172.1, 157.2 (2×C═O), 137.2, 129.2, 128.3, 127.7, 127.5, 126.6 (C arom), 101.6 (C-1), 71.3, 70.8, 70.7, 67.5 (C-2, C-3, C-4, C-5), 66.3 (CH₂ benzyl group), 64.8 (C-6), 55.9 (CH amino acid), 54.1 (OCH₃), 37.4 (CH₂ amino acid); MS (ES⁺): m/z=498 (100, [M+Na]⁺); HRMS (ES⁺): C₂₄H₃₃O₉N₂ calculated 493.2186; measured 493.2182 [M+NH4]+.

SBL-Catalyzed Carboydrate Selective Acylations from within Mixtures

12a (0.56 mmol), 16a (0.56 mmol), 1c (1.6 eq.), and pH adjusted SBL preparation (10 mg) were suspended in 4 mL of anhydrous pyridine and stirred under nitrogen at 45° C. for 7 days. The mixture was then concentrated and the residue purified by flash chromatography (CHCl₃:MeOH:AcOH:H₂O, 85:10:0.5:1) to 16b (80%). The starting material 12a was recovered as a second fraction.

19a (0.56 mmol), 20a (0.56 mmol), 1c (1.6 eq.), and pH adjusted SBL preparation (10 mg) were suspended in4 mL of anhydrous pyridine and stirred under nitrogen at 45° C. for 168 days. The mixture was then concentrated and the residue purified by flash chromatography (CHCl₃:MeOH:AcOH:H₂O, 85:10:0.5:1) to 19b (47%). The starting material 20a was recovered as a second fraction.

### EXAMPLE 2

### EXAMPLE 2A

### Construction of Pyranoside-Peptide Conjugates

In our project, we were interested in the use of proteases as typically robust and flexible enzymes. For example, in 1988, Klibanov (Riva et al (1988) described the use of Bacillus subtilis protease (subtilisin). This commercial enzyme was stable and active in numerous anhydrous organic solvents (pyridine, DMF), which were needed to dissolve the free sugars. We have chosen to use the Subtilisin of Bacillus lentus (SBL) to perform many of our enzymatic acylations. In 1998, Jones (Lloyd et al (1998)) described the use of this enzyme; it was shown that this enzyme was very useful in the catalysis of transesterifications between vinyl esters and different alcohols in good to excellent yields.

The effect of varying the amino acid acyl donor was investigated. Consistent with the observed low affinity of SBL for other amino acid esters (Khumtaveepom (1999)), none of the aspartate or glutamate acyl donors were readily accepted as substrates by SBL. In all cases only vinyl esters 4-8c were recovered indicating an absence of productive binding by SBL to form acyl-enzyme intermediate. This contrasted with the reactions of 1c from which only transesterification or hydrolysis products were recovered, thereby indicating ready formation of acyl-enzyme intermediate prior to reaction with carbohydrate alcohol or water respectively.

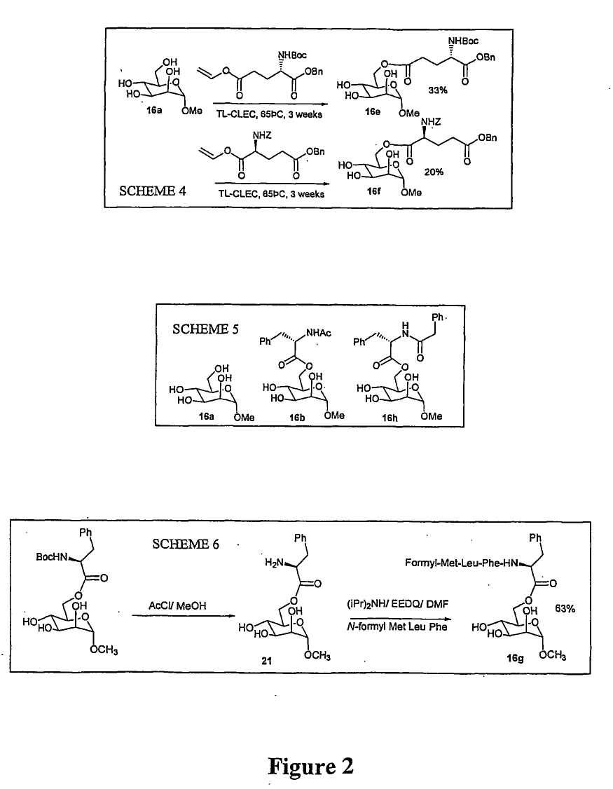

In order to further assess the utility of 1,4-8c as acyl donor probes, we also screened their reactivity with CLEC-thermolysin (TL-CLEC) as a protease with a different substrate specificity profile, that includes β-aspartate esters (Miyanaga et al (2000)). Initially, at 45° C., TL-CLEC also failed to readily accept 4-8c and again only 1c was accepted, allowing the preparation of 16b from 16a in 48% yield. However, prolonged reaction times and elevated temperatures (65° C.) pleasingly yielded corresponding 6-O-β-aspartate esters of mannoside 16e (33%) and α-aspartate ester 16f (40-55%) (see FIG. 2, Scheme 4). Compounds 16e,f are interesting building blocks to obtain other tethered derivatives since after deprotection the amino group can be reacted with other (glyco)peptide building blocks.

Next, the effect and manipulation of N-protection in the acyl donor was investigated (Boc- and Z-protected phenylalanine donors with 16a had already been investigated in Example 1). Differently cleaved N-protecting groups on the amino acid increase flexibility in coupling strategies and we prepared the methyl-6-O-carboxyl-(N-phenylacetyl-L-phenylalanine)-α-D-mannopyranoside compound 16h through use of the corresponding phenacetyl-protected Phe vinyl ester acyl donor with SBL in 57% yield. It is possible to remove the phenacetyl amino-protecting group enzymatically and hence under mild conditions (using penicillin G acylase PGA) (Waldmann et al (1996)). The utility of N-Boc protected compound methyl-6-O-carboxyl-(N-tert-butyloxycarbonyl-L-phenylalanine)-α-D-mannopyranoside as a glycopeptide building block was also confirmed through quantitative N-deprotection to methyl 6-O-phenylalaninyl-α-D-mannopyranoside 21, which was extended at its N-terminus. The deprotection of the amino group with AcCl/methanol (Nudelman et al (1998)) followed by peptidic coupling (EEDQ/DMF) with the commercial tripeptide N-formyl-Met-Leu-Phe, a chemotactic peptide, gave the derivative 16g (See FIG. 2, scheme 6). This may be viewed as an example of a potential prodrug N-formyl-Met-Leu-Phe-Phe-Man-α-OMe 16g of a biologically-active peptide N-formyl-Met-Leu-Phe. For example, the prodrug 16g may facilitate delivery in vivo through interaction with the mannose receptor before cleavage of the labile Phe-Phe or Phe-Man bonds, enzymatically or chemically, to yield active tripeptide N-formyl-Met-Leu-Phe.

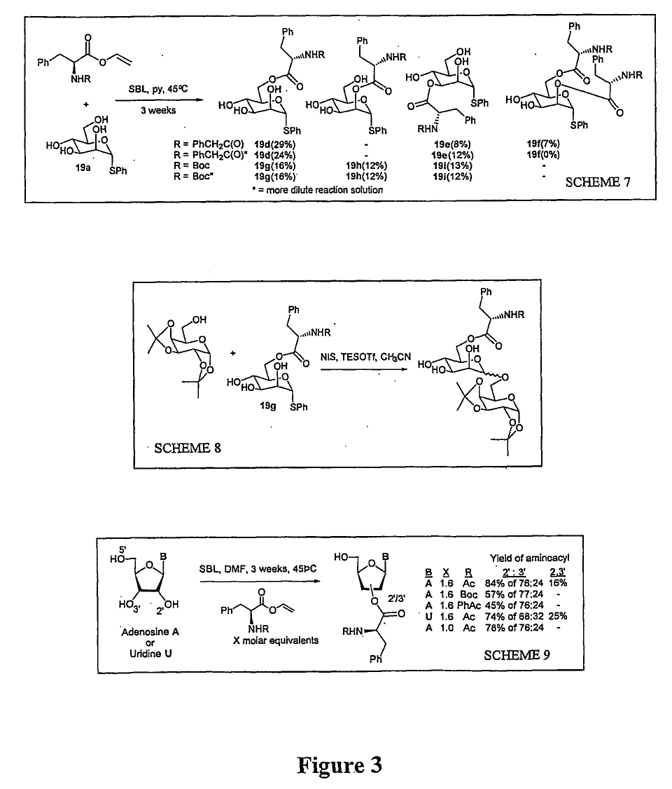

The acylation of glycosyl donor sugars with PheNHAc acyl donor 1c had been investigated above in Example 1. PheNHBoc and PheNHPhAc acyl donors for acylation of glycosyl donor thiophenyl mannopyranoside 19a were also investigated. Interestingly, low regioselectivity was observed and three regioisomers observed for both systems (see FIG. 3, scheme 7). Higher selectivity was observed in certain cases with more dilute enzymatic acylation conditions.

To demonstrate the utility of glycosylation, diacetoneglycosylation was reacted with 19g activated by N-iodosuccinimide/triethylsilyltriflate in acetonitrile (see FIG. 3, scheme 8) (Veeneman et al (1990) and Konradsson et al (1990)) This is a rare example of glycosylation with unprotected glycosyl donors (Hannessian et al (2000)).

### EXAMPLE 2B

### Generation of Nucleoside-Peptide Conjugates:

The powerful selectivity observed for pyranoside-peptide conjugation prompted us to investigate the use of SBL protease in furanoside, and in particular, riboside and ribonucleoside-peptide formation. We reasoned that, in particular, this may have utility in the formation of the CCA-peptide moiety of aminoacyl-tRNAs. The synthesis of these structures is complicated by the presence on the 2/3′ terminal hydroxyl groups of an amino acid-ester linkage, which renders them inaccessible by normal automated nucleic acid synthesis techniques.

Standard chemical methods or the use of other enzyme systems in riboside acylation gives 5′-O-acyl derivatives of no utility in acyl-tRNA synthesis. Indeed, in the literature, few examples of regioselective aminoacylation of nucleosides have been achieved. These have used standard chemical methods employing activated amino acids in pyridine (Oliver et al (1996)) or the Mitsunobu reaction (Montero et al (1991)), and necessitated protection steps. Alternatively, biocatalysis, and more specifically the use of lipases (Moris and Gotor (1993), Ciuffreda et al (1999) and Gotor and Moris (1992)), subtilisin (Riva et al (1988)) or mutant subtilisin (Wong et al (1990)), allowed the creation of regioselectively amiinoacylated nucleosides in one step. In such biocatalytic steps, the 5′-O-acyl derivatives have always been the major product.

However, applying the methodology described above reaction of adenosine (A) or uridine (U) with protected Phe vinyl esters allowed the synthesis of exclusively 2′/3′-acylated products. To our knowledge this is the first example of absolute OH-2′/3′ over OH-5′ acylation selectivity (Ferrero and Gotor (2000) and Prasad and Wengel (1996)). Thus, using SBL in DMF at 45° C. with adenosine (A) or uridine (U), we never observed the acylation of OH-5′ (see FIG. 3, scheme 9). The use of pyridine led to no reaction. As a result of rapid intramolecular O-2′⇄O-3′ acyl migration the O-3′ and O-2-acylated products are in equilibrium and were not separated. Under physiological conditions this equilibrium allows both products to serve as sources of acylated ribonucleoside. Variation of the N-protecting group on the Phe was also possible using this system (Boc, PhAc or Ac) thereby opening up routes to the formation of N-unprotected acyl-tRNA precursors.

The unusual O-2′/3′ acylated structures were confirmed by carefuil characterization and NMR chemical shift/HMBC analysis (data not shown). Specifically, chemical shifts in ¹H and ¹³C NMR for the starting materials and the compounds formed were compared. A Δδ shift for the protons H-2 or H-3 and a shift for the carbons C-2 or C-3 was observed consistent with acylation of OH-2′/3′. No significant shifts were observed for the H-5 or C-5 except for the O5′-acylated methyl-β-D-ribofuranoside (as expected). To confirm the structures of the acylated compounds formed, HMBC NMR experiments were also carried out. For each compounds, we observed a correlation between the carbonyl function of the amino acid and the H-2′ or H-3′ proton.

The mechanism of this intriguing regioselective aminoacylation was investigated by detailed NMR analysis. An enzymatic reaction between adenosine and N-acetyl-L-phenylalanine vinyl ester in d₆-DMF was observed at 300 MHz for one week. In this system one can easily follow the decrease of the starting material (δ H-3=4.32 ppm) and the formation of O-3′-acylated (δ H-2=5.07 ppm) and O-2′-acylated (δ H-1=6.32 ppm) products. This revealed initial acylation of OH-2′, followed by the known (Reese and Trentham (1965)) intramolecular migration from OH-2′ to OH-3′.

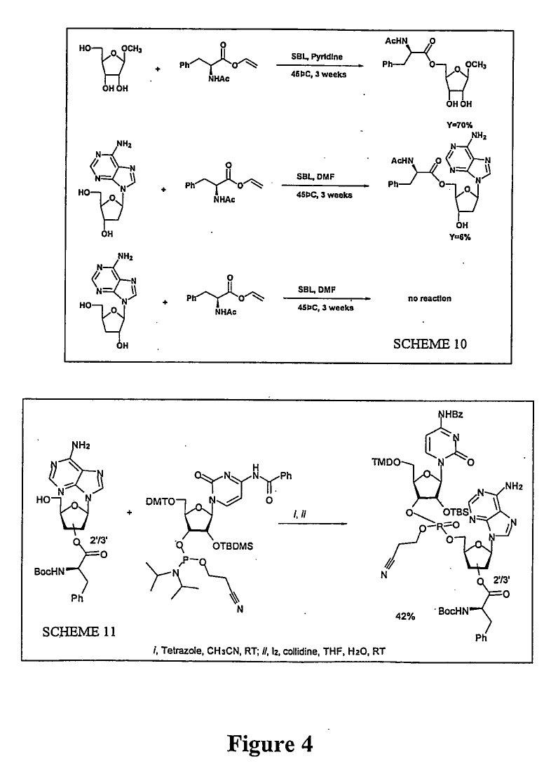

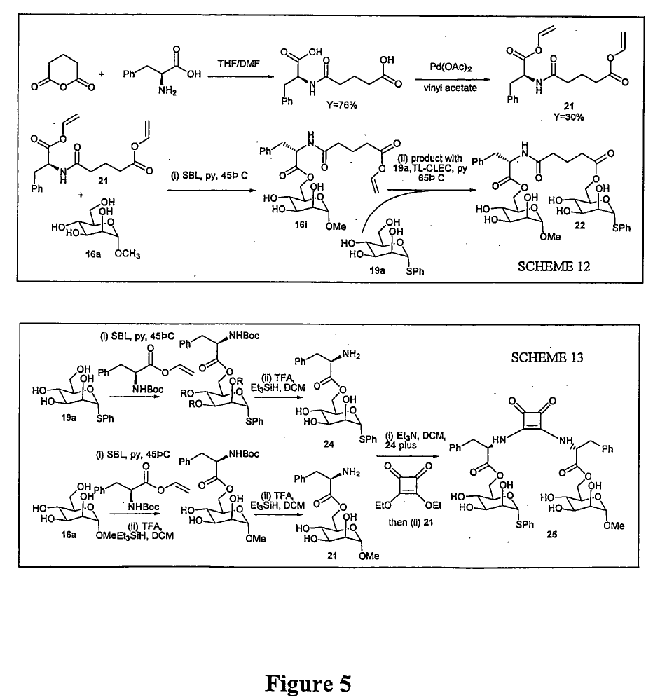

Intriguingly, SBL transferred the N-acetyl-L-phenylalanine vinyl ester exclusively to the primary alcohol (OH-5′) of methyl D-riboside in 70% yield (see FIG. 4, scheme 10) 3′-deoxyadenosine did not react and from the reaction with 2′-deoxyadenosine we isolated the 5′-O-acylated-2′-deoxyadenosine product in 6% yield. The loss of either of the hydroxyl groups (3′-OH or 2′-OH) or the base from the anomeric centre dramatically changed the reaction outcome. Thus, the valuable 2′/3′ over 5′ selectivity is uniquely dependent on the use of the Phe acyl donors and nucleosides U or A in combination with SBL under the conditions that we have elucidated.

A subsequent, successful regioselective (selective phosphitylation of the primary 5′-OH) phosphoramidite-coupling strategy & I₂-mediated oxidation (Barone et al (1984)) gave anunoacyl CA-dinucleotides as acyl-tRNA precursors (see FIG. 4, scheme 11). Notable is the selective phosphitylation of primary 5′-OH over the 2′ or 3′-OH of the aminoacylated adenosinyl residue. The structure of the dinucleotides were confirmed by ¹H, ³¹P and ¹³C NMR experiments. This entire route remarkably avoided the need for protection of the adenosinyl residue. With this key methodology in place, the total synthesis of an acyl-tRNA should be possible. Key deprotections using TFA allowed DMT removal and using CAN (fwu et al (1996)) to remove DMT and Boc to illustrate the flexibility to create acyl-tRNA precursors. In addition the use of N-phenacetyl protection allowed the formation of a corresponding PhAc protected dinucleotide.

The dramatic innovations of solid phase oligonucleotide synthesis by Khorana and Carruthers revolutionized approaches to gene synthesis. The strategy of choice for such syntheses involves 3′ to 5′ linear assembly residue-by-residue and is applicable to nearly all oligonucleotides of interest. Notable exceptions are the aminoacyl-tRNAs, vital components in protein biosynthesis, whose synthesis is complicated by the presence on the 2/3′ terminal hydroxyl groups of an often-labile amino acid-ester linkage. Mimetics of the key CCA-[aa] acyl-tRNA motif have been accessed in limited strategies that use ligase-catalyzed ligation of a dCA-[aa] (or dCdCA-[aa]) moiety to a truncated tRNA, to create an aminoacyl-tRNA mimic in which hydroxyl groups in the 3′-CCA terminus, and which may influence the interactions of tRNA, are missing. The direct acylation of tRNA and hence the only true synthesis of aminoacyl-tRNA is achievable through the in vivo use of tRNA-synthetases, expensive and limited enzymes. More recently, a ribozyme was used to exclusively aminoacylate the 3′-hydroxyl group of the tRNA terminal adenosine (Saito and Suga (2001)). Here we exploited our rare 3′ over 5′ hydroxyl (secondary over primary) aminoacylation to create a CA-[aa] building block, without the use of protection, suitable for a 3′ to 5′ aminocyl-tRNA total synthesis strategy.

Thus in summary, a remarkable and rare 2/3′ over 5′ hydroxyl (secondary over primnary) aminoacylation to create an A-[aa] building block, without the use of protection, suitable for a 3′ to 5′ aminocyl-tRNA total synthesis strategy was discovered as an extension to investigations in carbohydrate-peptide conjugate construction.

### EXAMPLE 2C

### The Use of Regioselective Acylation to Construct Peptide-Bridged Carbohydrates of Potential Use in Tethered Oligosaccharide Synthesis:

To survey other potential enzyme systems a screen of 28 acyltransferases for their ability to perform esterfications and their robustness to organic solvents was carried out and confirmed that the use of SBL and TL-CLEC give the highest yielding results in peptide-carbohydrate ester formation strategies. With this information in hand two templated glycosylation strategies were devised:

(i) link donor and acceptor carbohydrate via enzyme-catalyzed ester formation to a pre-formed peptide based template. Selectivity was obtained by having a L-phenylalaninyl moiety at one terminus of the template (for recognition by one enzyme (e.g. SBL) and an orthogonal (to be recognised by another enzyme e.g. TL-CLEC) vinyl ester moiety at the other; and

(ii) to tether C-6 linlced glycoamino acid esters obtained from the method of Example 2A above together directly using diethyl squarate of another bifunctional amine-reactive linker.

(i) Esterification onto a Pre-Formed Peptide-Based Template:

For strategy (i) peptide-based linker 21 was constructed from phenylalanine and glutaric anhydride followed by Pd(OAc)₂-mediated transesterification to create the bis vinyl ester 21. After careful optimisation of conditions, a remarkable two-step process to tether mannoside glycosyl acceptor 16a to mannoside glycosyl donor 19a was achieved using a two transesterification sequential SBL-mediated then TL-CLEC-mediated catalysed process to yield peptide-bridged complex 22 via 16i.

It should be noted that despite the modest yield, to the best of our lknowledge, this is only the second ever regioselective tethering reaction of its kind (KhmeInitsky (1997)) and the first for linking carbohydrate-to-carbohydrate. 22 is of potential tethered oligosaccharide synthesis.

(ii) Esterification and then Elaboration to a Peptide-Based Template:

Strategy (b) utilized building blocks generated in Example 2A to construct tethered system 25 through sequential reaction of 24 and 21 with diethyl squarate. This was achieved by SBl-mediated acylation of 16a with PheNHBoc followed by deprotection to give 21. The parallel reaction was performed for thioglycoside 19a to yield 24. The utility of this system in tethered glycosylation is currently under investigation.

In summary, the high specificity of our developed regioselective acylation methodology was used as a tool to create carbohydrates tethered to each other via a peptide-containing bridge in peptide templated glycosylation strategies (Tennant-Eyles et al (1999) and Jung et al (2000). The enzyme was used to selectively esterify sugar alcohols with N-protected phenylalanine residues which either contained a second ester for tethering or were then elaborated into peptide-templated carbohydrate-carbohydrate systems of potential use in tethered oligosaccharide synthesis. The regioselectivity of SBL for OH-6 allowed use of minimally-protected starting materials thereby removing much of the complicated protecting group manipulations that plague most (including tethered intramolecular syntheses) oligosaccharide syntheses.

Supplementary Information

Methyl-6-O-carboxy-(benzyl-N-tert-butyloxycarbonyl-L-glutamate)-α-D-mannopyranoside 16e

Methyl-α-D-mannopyranoside 16e (200 mg; 1.03 mmol), benzyl β-vinyl-N-(t-butyloxycarbonyl)-L-glutamate (374 mg; 1 eq.) and 2 mg of thermolysin TL-CLEC were suspended in anhydrous pyridine (5 mL) and stirred under nitrogen at 65° C. for 3 weeks. The reaction was filtered through celite, evaporated and the residue purified by flash chromatography (ethyl acetate/methanol 100/0.5 v/v) to give 33a (100 mg; 20%) after lyophilisation: [α]_{D}²⁵=+8.4 (c=0.25 in methanol); IR ν max (KBr): 3392 cm⁻¹ (OH, NH); 1740 cm⁻¹ (CO); 1522 cm⁻¹ (amide I); 1546 cm (amide II); ¹³C NMR (125 MHz, CD₃OD) δ 174.2-173.6 (3×C═O); 137.2-137.1-129.5-129.2-129.16-127.13 (C arom); 102.6 (C-1); 80.6 (C Boc); 72.4-71.83-71.80-68.6 (C-2; C-3; C-4; C-5); 67.9 (CH₂ benzyl); 65.1 (C-6); 55.2 (OCH₃); 54.4 (CH amino acid); 31.3 (CH₂ amino acid); 28.7 (3×CH₃); (27.5 (CH₂); ¹H NMR (500 MHz, CD₃OD) δ 7.42-7.31 (m; 5H; 5H arom); 5.18 (m; 2H; CH₂ benzyl); 4.63 (d; J 1.4 Hz; 1H; H-1); 4.44 (d; J 11.3 Hz; 1H; H-6); 4.26 (m; 2H; H-6′ and H amino acid); 3.82 (m; 1H; H-2); 3.67 (m; 3H; H-5; H-4; H-3); 3.34 (s; 3H; OCH₃); 2.47 (m; 2H; CH₂ amino acid); 2.18 (m; 1H; CH₂ amino acid); 1.96 (m; 1H; CH₂ amino acid); 1.44 (s; 9H; 3×CH₃); MS (ES+) m/z=514 [M+H]⁺; 536 [M+Na]⁺; 552 [M+K]⁺; HRMS (ES⁺): calculated 536.2108; measured 536.2111 [M+Na].

Methyl-6-O-carboxy-(benzyl-N-benzyloxycarbonyl-L-glutamate)-α-D-mannopyranoside 16f

16e (25 mg; 0.13 mmol), benzyl α-vinyl-N-(t-butyloxycarbonyl)-L-glutamate (50 mg; 1 eq.) and 2 mg of thermolysin TL-CLEC were suspended in anhydrous pyridine (2 mL) and stirred under nitrogen at 65° C. for 3 weeks. The reaction was filtered through celite, evaporated and the residue purified by flash chromatography (chloroform/methanol/acetic acid/water 100/5/1/0.5 v/v) to give 16f (15 mg; 20%) after lyophilisation.

Glycotetrapeptide 16g

Methyl-6-O-carboxy-(N-tert-butyloxycarbonyl-L-phenylalanine)-α-D-mannopyranoside was dissolved in methanol (1 mL) and the acetylchloride (10 μL) was slowly added at 0° C. After warm up at room temperature, the mixture was concentrated in vacuo. The residue was dissolved in DMF and diisopropylethylamine (30 μL; 4 eq.) was added. After 1 h at room temperature EEDQ (15 mg; 1 eq.) was added and the mixture was stirred for 1 h at room temperature. Commercially available (Sogma) N-formyl-Met-Leu-Phe tripeptide (20 mg; 1 eq.) was then added. After 12 h, the mixture was concentrated in vacuo and purified by flash chromatography (chloroform/methanol/water/acetic acid 85/10/11/0.5 v/v) to give 16g (63%): MS (ES⁺): m/z=783 [M+Na]⁺; HRMS (ES⁺): calculated 761.3431; measured 761.3433 [M+H].

Vinyl N-(phenylacetyl)oxycarbonyl-L-phenylalaninate

L-phenylalanine (2 g; 12 mmol) was dissolved in an aqueous solution of sodium hydroxide (1N; 25 mL). Then, phenyl acetyl chloride (1.9 mL; 1.2 eq.) in solution in dioxane (8 mL) was slowly added at 0° C. After one night, the mixture was neutralized with an aqueous solution of HCl (1N), and extracted with ethyl acetate (3 times). The organic layers were dried over sulfate magnesium and concentrated in vacuo to give a white solid.

The crude acid was dissolved in vinyl acetate (110 mL; 100 eq.), then palladium acetate (539 mg; 0.2 eq.) and potassium hydroxide (67 mg; 0.1 eq.) were added. The mixture was stirred overnight at room temperature, then poured into diethyl ether and filtered through a celite bed. After concentration in vacuo, the crude product was purified by flash chromatography (hexane/ethyl acetate 4/1 then 2/1 v/v) to give vinyl N-(phenylacetyl)oxycarbonyl-L-phenylalaninate as a yellow oil (1.7 g; 45% over two steps): [α]_{D}²⁵=+2.2 (c=1.1 in chloroform); IR ν max (film): 3269 cm⁻¹ (NH), 1763 cm⁻¹ (C═O), 1667 cm⁻¹ (C═O), 1529 cm⁻¹ (amide I), 1454 cm⁻¹ (amide II); ¹³C NMR (125 MHz, CDCl₃) δ 171.0-168.9 (2×CO); 141.0-140.9-135.4-134.5-129.7-129.4-129.3-128.9-127.7-127.4 (10×C arom and CH vinyl group); 99.4 (CH₂ vinyl group); 53.0 (CH); 43.7 (CH₂ benzyl group); 37.5 (CH₂); ¹H NMR (500 MHz, CDCl₃) δ 7.36-7.27 (m; 4H; H arom); 7.21-7.18 (m; 5H; 4H arom and CH vinyl group); 6.91-6.90 (m; 2H; H arom); 5.98 (dl, J 8.1 Hz; 1H; NH); 4.94 (dd; J 2.0 Hz; J 13.8 Hz; 1H; H vinyl group); 4.93 (m; 1H; CH); 4.67 (dd; J 1.8 Hz; J 6.2 Hz; 1H; H vinyl group); 3.57 (s; 2H; CH₂ benzyl group); 3.11 (dd; J 5.6 Hz; J 14.1 Hz; 1H); 3.05 (dd; J 5.6 Hz; J 14.1 Hz; 1H); MS (ES+) m/z 332 [M+Na]⁺; HRMS (ES⁺): calculated 310.1443; measured 310.1442 [M+H].

Methyl-6-O-carboxy-(N-phenylacetyloxycarbonyl-L-nhenylalanine)-α-D-mannopyranoside 16h

Methyl-α-D-mannopyranoside (50 mg; 0.26 mmol), N-phenylacetyl-phenylalanine vinyl ester (127 mg; 1.6 eq.) and 50 mg of pH adjusted SBL preparation were suspended in 7 mL of anhydrous pyridine and stirred under nitrogen at 45° C. for 5 days. The reaction was filtered through celite, evaporated and the residue purified by flash chromatography (chloroform/methanol/acetic acid/water 90/10/0.5/1) to give 16 h (68 mg; 57%) after lyophilisation: [α]_{D}²⁵=+30.9 (c=0.89 in methanol); IR: νₘₐₓ (KBr): 3410 cm⁻¹ (OH, NH); 1741 cm⁻¹ (C═O); 1653 cm⁻¹ (amide I); 1497 cm⁻¹ (amide II); ¹H NMR (500 MHz; CD₃OD): δ 7.25-7.14 (m; 10H; H arom); 4.78 (dd; J 4.8 Hz; J 9.4 Hz; 1H; H c); 4.63 (d; J 1.6 Hz; 1H; H-1); 4.45 (dd; J 2.3 Hz; J 11.6 Hz; 1H; H-6); 4.32 (dd; J 6.3 Hz; J 11.2 Hz; 1H; H-6′); 3.81 (dd; J 1.8 Hz; J 3.1 Hz; 1H; H-2); 3.70-3.60 (m; 3H; H-3, H-4 and H-5); 3.48 (s; 2H; CH₂—Ph); 3.34 (OCH₃); 3.23 (dd; J 5.5 Hz; J 14.3 Hz; 1H; H′ β); 2.98 (dd; J 9.1 Hz; J 13.9 Hz; 1H; H′ β); ¹³C NMR (125.7 MHz; CD₃OD): δ 173.8-172.8 (2×CO); 138.0-127.8 (C arom); 102.8 (C-1); 72.5-71.9-71.8-68.6 (C-2; C-3; C-4; C-5); 65.9 (C-6); 55.3 (OCH₃); 55.1 (CH amino acid); 43.4 (CH₂-Ph); 38.2 (CH₂ amino acid); MS (ES⁺): m/z=482 [M+Na]⁺; HRMS (ES⁺): calculated 477.2237; measured 477.2330 [M+NH₄]+.

Enzymatic Acylation of Adenosine of with N-acetyl-L-phenylalanine Vinyl Ester & SBL

Adenosine (250 mg; 0.93 mmol), N-acetyl-L-phenylalanine vinyl ester (350 mg; 1.6 eq.) and 30 mg of pH adjusted SBL preparation were suspended in 10 mL of anhydrous DMF and stirred under nitrogen at 45° C. for 3 weeks. The reaction was then concentrated in vacuo and the residue purified by flash chromatography (chloroform/methanol/acetic acid/water 100/3/0.5/1 v/v) to give after lyophilisation diacyl 2′,3′ (98 mg; 16%) and a mixture of 2′/3′ (370 mg; 84%; ratio 3′′:2′=76:24): 2,3-Di-O-carboxy-(N-acetyl-L-phenylalanine)-adenosine 19a: [α]_{D}²⁵=−56.6 (c=0.14 in methanol); IR νₘₐₓ (KBr): 3423 cm⁻¹ (OH, NH); 1752 cm⁻¹ (C═O); 1654-1603 cm⁻¹ (amide I); 1547 cm⁻¹ (amide II); ¹H NMR (500 MHz; CD₃OD): δ□8.24 (s; 1H; CH double bond); 8.20 (s; 1H; CH double bond); 7.33-7.15 (m; 10H; H arom); 6.10 (d; J7.3 Hz; 1H; H-1); 5.94 (dd; J5.3 Hz; J7.7 Hz; H-2); 5.56 (dd; J 1.7 Hz; J 5.4 Hz; 1H; H-3); 4.81 (t; J 7.6 Hz; 1H; H α); 4.59 (dd; J 5.2 Hz; J 9.9 Hz; 1H; H α); 3.94 (m;1H; H-4); 3.82 (dd; J 2.5 Hz; J 12.9 Hz; 1H; H-5); 3.70 (dd; J 2.5 Hz; J 12.7 Hz; 1H; H-5′); 3.13 (m; 3H; 3×H′ β); 2.92 (dd; J 10.2 Hz; J 14.5 Hz; 1H; H′ β); 1.90 (s; 3H; NHCOCH₃); 1.82 (s; 3H; NHCOCH₃); ¹³C NMR (125.7 MHz; CD₃OD): δ 172.0-171.9 (2×NHCOCH₃); 171.3 (CO); δ 170.5 (CO); 156.5 (C arom); 152.5 (CH double bond); 148.9 (C arom); 140.6 (CH double bond); 137.0 to 126.7 (C arom); 120.0 (C—NH₂); 86.9 (C-1); 85.0 (C-4); 73.7 (C-2); 73.1 (C-3); 61.9 (C-5); 54.3-53.8 (2×CH amino acid); 37.6-36.6 (2×CH₂ amino acid); 21.2-21.0 (2×NHCOCH₃); MS (ES⁺): m/z=646 [M+H]⁺; 668 [M+Na]⁺; HRMS (ES⁺): calculated 646.2625; measured 646.2610 [M+H].

3-O-carboxy-(N-acetyl-L-phenylalanine)-adenosine: [α]_{D}²⁵=−41.4 (c=0.34 in methanol); IR ν max (KBr): 3347 cm⁻¹ (OH, NH); 1748 cm⁻¹ (C═O); 1648-1604 cm⁻¹ (amide I); 1559 cm⁻¹ (amide II); ¹H NMR (500 MHz; CD₃OD): δ 8.30 (s; 1H; CH double bond); 8.20 (s; 1H; CH double bond); 7.35-7.16 (m; 5H; H arom); 5.88 (d; J 7.4 Hz; 1H; H-1); 5.38 (dd; J 1.5 Hz; J 5.3 Hz; 1H; H-3); 4.99 (dd; J 5.5 Hz; J 7.3 Hz; 1H; H-2); 4.84 (dd; J 6.8 Hz; J 8.7 Hz; 1H; H′ α); 4.02 (m; 1H; H4); 3.83 (dd; J 12.7 Hz; J 2.3 Hz; 1H; H-5); 3.71 (dd; J 12.8 Hz; J 2.2 Hz; 1H; H-5′); 3.22 (dd; J 6.6 Hz; J 13.7 Hz; 1H; H′ β); 3.10 (dd; J 8.7 Hz; J 14.4 Hz; 1H; H′ β); 1.97 (s; 3H; NHCOCH₃); ¹³C NMR (125.7 MHz; CD₃OD): δ 172.1 (NHCOCH₃); 171.2 (CO); 156.5 (C arom); 152.4 (CH double bond); 148.9 (C arom); 140.8 (CH double bond); 136.9 to 126.7 (C arom); 119.9 (C—NH₂); 89.5 (C-1); 84.6 (C-4); 74.9 (C-3); 72.8 (C-2); 62.1 (C-5); 54.3 (CH amino acid); 37.5 (CH₂ amino acid); 21.1 (NHCOCH₃); MS (ES⁺): m/z=479 [M+Na]⁺; HRMS (ES⁺): calculated 457.1835; measured 457.1831 [M+H].

2-O-carboxy-(N-acetyl-L-phenylalanine)-adenosine: [α]_{D}²⁵=41.4 (c=0.34 in methanol); IR νₘₐₓ (KBr): 3347 cm⁻¹ (OH, NH); 1748 cm⁻¹ (C═O); 1648-1604 cm⁻¹ (amide I); 1559 cm (amide II); ¹H NMR (500 MHz; CD₃OD): δ 8.30 (s; 1H; CH double bond); 8.20 (s; 1H; CH double bond); 7.35-7.16 (m; 5H; H arom); 6.23 (d; J 6.1 Hz; 1H; H-1); 5.69 (t; J 5.5 Hz; 1H; H-2); 4.70 (dd; J 5.4 Hz; J 9.2 Hz; 1H; H′ α); 4.66 (dd; J 3.4 Hz; J 5.4 Hz; 1H; H-3); 4.21 (m; 1H; H-4); 3.93 (dd; J 12.6 Hz; J 2.5 Hz; 1H; H-5); 3.77 (dd; J 12.6 Hz; J 2.5 Hz; 1H; H-5′); 3.22 (m; 1H; H′ β); 2.95 (dd; J 9.4 Hz; J 14.4 Hz; 1H; H′ β); 1.87 (s; 3H; NHCOCH₃); ¹³C NMR (125.7 MHz; CD₃OD): δ 172.1 (NHCOCH₃); 170.7 (CO); 156.5 (C arom); 152.5 (CH double bond); 148.9 (C arom); 140.8 (CH double bond); 136.9 to 126.7 (C arom); 119.9 (C—NH₂); 87.4 (C-1); 87.3 (C-4); 76.6 (C-2); 69.9 (C-3); 61.9 (C-5); 54.1 (CH amino acid); 36.9 (CH₂ amino acid); 21.0 (NHCOCH₃); MS (ES⁺): m/z=479 [M+Na]⁺; HRMS (ES⁺): calculated 457.1835; measured 457.1831 [M+H].

Enzymatic Acylation of Adenosine of with N-Boc-L-phenylalanine Vinyl Ester &SBL

Adenosine (500 mg; 1.8 mmol), N-tert-butyloxycarbonyl-L-phenylalanine vinyl ester (871 mg; 1.6 eq.) and 30 mg of pH adjusted SBL preparation were suspended in 10 mL of anhydrous DMF and stirred under nitrogen at 45° C. for 3 weeks. The reaction was then concentrated in vacuo and the residue purified by flash chromatography (chloroform/methanol/acetic acid/water 100/3/0.5/1 v/v) to give after lyophilisation a mixture of 2′-O-acyl and 3′-O-acyl (550 mg; 57%; ratio 3′:2′=77:23):

3-O-carboxy-(N-tert-butyloxycarbonyl-L-phenylalanine)-adenosine: [α]_{D}²⁵=−64.2 (c=1.36 in methanol); IR νₘₐₓ (KBr): 3348 cm⁻¹ (OH, NH); 1754-1716 cm⁻¹ (C═O); 1656-1612 cm⁻¹ (amide I); 1580-1500 cm⁻¹ (amide II); ¹H NMR (500 MHz; CD₃OD): δ8.29 (s; 1H; CH double bond); 8.19 (s; 1H; CH double bond); 7.32-7.14 (m; 5H; H arom); 5.89 (d; J 7.6 Hz; 1H; H-1); 5.37 (dd; J 1.1 Hz; J 5.3 Hz; 1H; H-3); 4.97 (dd; J 5.8 Hz; J 7.3 Hz; 1H; H-2); 4.52 (dd; J 6.6 Hz; J 8.8 Hz; 1H; H′ cc); 4.02 (m; 1H; H-4); 3.82 (dd; J 12.8 Hz; J 2.3 Hz; 1H; H-5); 3.71 (dd; J12.8 Hz; J2.3 Hz; 1H; H-5′); 3.17 (dd; J7.2 Hz; J 13.5 Hz; 1H; H′ β); 3.04 (dd; J9.3 Hz; J 13.9 Hz; 1H; H′ β); 1.40 (s; 9H; C(CH₃)₃); ¹³C NMR (125.7 MHz; CD₃OD): δ 173.0 (CO); 172.4 (NHCOCH₃); 157.9-157.6 (2×C arom); 153.6 (CH double bond); 150.0 (C arom); 141.9 (CH double bond); 138.3 to 127.9 (C arom); 121.0 (C—NH₂); 90.6 (C-1); 85.8 (C-4); 80.8 (C(CH₃)₃); 75.9 (C-3); 73.9 (C-2); 63.3 (C-5); 56.8 (CH amino acid); 38.9 (CH₂ amino acid); 28.7 (C(CH₃)₃); MS (ES⁺): m/z=537 [M+Na]⁺; HRMS (ES⁺): calculated 515.2254; measured 515.2246 [M+H].

2-O-carboxy-(N-tert-butyloxycarbonyl-L-phenylalanine)-adenosine: [α]_{D}²⁵−64.2 (c=1.36 in methanol); IR νₘₐₓ (KBr): 3348 cm⁻¹ (OH, NH); 1754-1716 cm⁻¹ (C═O); 1656-1612 cm⁻¹ (amide I); 1580-1500 cm⁻¹ (amide II); ¹H NMR (500 MHz; CD₃OD): δ 8.29 (s; 1H; CH double bond); 8.19 (s; 1H; CH double bond); 7.32-7.14 (m; 5H; H arom); 6.21 (d; J 6.6 Hz; 1H; H-1); 5.68 (t; J 5.2 Hz; 1H; H-2); 4.64 (dd; J 3.4 Hz; J 5.3 Hz; 1H; H-3); 4.40 (dd; J 5.2 Hz; J 8.8 Hz; 1H; H′ α); 4.20 (m; 1H; H-4); 3.90 (dd; J 12.4 Hz; J 2.3 Hz; 1H; H-5); 3.77 (dd; J 12.8 Hz; J 2.9 Hz; 1H; H-5′); 3.14 (dd; J 5.5 Hz; J 12.9 Hz; 1H; H′ β); 2.90 (dd; J 9.0 Hz; J 12.9 Hz; 1H; H′ β); 1.34 (s; 9H; C(CH₃)₃); ¹³C NMR (125.7 MHz; CD₃OD): δ 173.0 (CO); 172.4 (NHCOCH₃); 157.9-157.6 (2×C arom); 153.5 (CH double bond); 150.0 (C arom); 141.9 (CH double bond); 138.3 to 127.9 (C arom); 121.0 (C—NH₂); 88.6 (C-1); 88.4 (C-4); 80.8 (C(CH₃)₃); 77.6 (C-2); 71.1 (C-3); 63.1 (C-5); 56.5 (CH amino acid); 38.3 (CH₂ amino acid); 28.6 (C(CH₃)₃); MS (ES⁺): m/z=537 [M+Na]⁺; HRMS (ES⁺): calculated 515.2254; measured 515.2246 [M+H].

Enzymatic Acylation of Adenosine of with N-phenylacetyl-L-phenylalanine Vinyl Ester & SBL

Adenosine (500 mg; 1.8 mmol), N-phenylacetyl-L-phenylalanine vinyl ester (925 mg; 1.6 eq.) and 30 mg of pH adjusted SBL preparation were suspended in 10 mL of anhydrous DMF and stirred under nitrogen at 45° C. for 3 weeks. The reaction was then concentrated in vacuo and the residue purified by flash chromatography (chloroform/methanol/acetic acid/water 100/3/0.5/1 v/v) to give after lyophilisation a mixture of 3′-O-acyl and 2′-O-acyl (450 mg; 45%; ratio 3′:2′=76:24):

3-O-carboxy-(N-phenylacetyloxycarbonyl-L-phenylalanine)-adenosine: [α]_{D}²⁵=−69.4 (c=0.87 in methanol); IR νₘₐₓ (KBr): 3345 cm⁻¹ (OH, NH); 1754-1706 cm⁻¹ (C═O); 1652-1603 cm⁻¹ (amide I); 1576-1500 cm⁻¹ (amide II); ¹H NMR (500 MHz; CD₃OD): δ 8.27 (s; 1H; CH double bond); 8.21 (s; 1H; CH double bond); 7.30-7.09 (m; 5H; H arom); 5.86 (d; J 7.2 Hz; 1H; H-1); 5.04 (dd; J 1.7 Hz; J 5.4 Hz; 1H; H-3); 4.98 (dd; J 4.9 Hz; J 7.7 Hz; 1H; H-2); 4.85 (dd; J 6.2 Hz; J 8.9 Hz; 1H; H′ α); 4.02 (m; 1H; H-4); 3.82 (dd; J 12.6 Hz; J 2.5 Hz; 1H; H-5); 3.70 (dd; J 12.6 Hz; J 2.5 Hz; 1H; H-5′); 3.55 (s; 2H; CH₂Ph); 3.26 (dd; J 6.4 Hz; J 14.3 Hz; 1H; Hβ β); 3.11 (dd; J 9.3 Hz; J 14.3 Hz; 1H; H′ β); ¹³C NMR (125.7 MHz; CD₃OD): δ 172.8 (CO); 171.0 (NHCOCH₃); 156.5 (CH arom); 152.4 (CH double bond); 148.9 (C arom); 140.7 (CH double bond); 136.9 to 126.7 (C arom); 120.0 (C—NH₂); 89.5 (C-1); 84.6 (C4); 74.9 (C-3); 72.8 (C-2); 62.1 (C-5); 54.3 (CH amino acid); 42.3 (CH₂Ph); 37.3 (CH₂ amino acid); MS (ES⁺): m/z=555 [M+Na]⁺; HRMS (ES⁺): calculated 533.2149; measured 533.2147 [M+H].

2-O-carboxy-(N-phenylacetyloxycarbonyl-L-phenylalanine)-adenosine: [α]_{D}²⁵=−64.9 (c=0.87 in methanol); IR νₘₐₓ (KBr): 3345 cm⁻¹ (OH, NH); 1754-1706 cm⁻¹ (C═O); 1652-1603 cm⁻¹ (amide I); 1576-1500 cm⁻¹ (amide II); ¹H NMR (500 MHz; CD₃OD): δ8.24 (s; 1H; CH double bond); 8.20 (s; 1H; CH double bond); 7.30-7.09 (m; 5H; H arom); 6.17 (d; J 5.6 Hz; 1H; H-1); 5.70 (t; J6.5 Hz; 1H; H-2); 4.72 (dd; J5.1 Hz; J9.9 Hz; 1H; H′ α); 4.67 (dd; J3.3 Hz; J 5.4 Hz; 1H; H-3); 4.18 (m; 1H; H-4); 3.92 (dd; J 12.6 Hz; J 2.5 Hz; 1H; H-5); 3.78 (dd; J 12.6 Hz; J2.5 Hz; 1H; H-5′); 3.44 (s, 2H; CH₂Ph); 3.22 (dd; J 5.1 Hz; J 12.8 Hz; 1H; H′ β); 2.96 (dd; J 9.5 Hz; J 13.7 Hz; 1H; H′ β); ¹³C NMR (125.7 MHz; CD₃OD): 6172.7 (CO); 170.6 (NHCOCH₃); 156.5 (C arom); 152.3 (CH double bond); 148.9 (C arom); 140.6 (CH double bond); 136.8 to 126.7 (C arom); 120.0 (C—NH₂); 87.3 (C-1); 87.2 (C-4); 76.6 (C-2); 69.8 (C-3); 61.9 (C-5); 54.0 (CH amino acid); 42.2 (CH₂Ph); 36.7 (CH₂ amino acid); MS (ES⁺): m/z=555 [M+Na]⁺; HRMS (ES⁺): calculated 533.2149; measured 533.2147 [M+H].

Enzymatic Acylation of Uridine with N-acetyl-L-phenylalanine Vinyl ester & SBL

Uridine (100 mg; 0.41 mmol), N-acetyl-L-phenylalanine vinyl ester (153 mg; 1.6 eq.) and 30 mg of pH adjusted SBL preparation were suspended in 5 mL of anhydrous DMF and stirred under nitrogen at 45° C. for 3 weeks. The reaction was then concentrated in vacuo and the residue purified by flash chromatography (chloroform/methanol/acetic acid/water 90/10/0.5/1 v/v) to give after lyophilisation diacyl (63 mg; 24%) and a mixture of 3′-O-acyl and 2′-O-acyl (140 mg; 75%; ratio 3′: 2′=68:32): 2,3-Di-O-carboxy-(N-acetyl-L-phenylalanine)-uridine: [α]_{D}²⁵=−14.8 (c=0.36 in methanol); IR νₘₐₓ (KBr): 3385 cm⁻¹ (OH, NH); 1755-1700 cm⁻¹ (C═O); 1664 cm⁻¹ (amide I); 1542 cm⁻¹ (amide II); ¹H NMR (500 MHz; CD₃OD): δ7.95 (d; J 8.0 Hz; CH double bond); 7.32-7.22 (m; 10H; H arom); 6.05 (d; J 5.8 Hz; 1H; H-1); 5.75 (d; J 7.7 Hz; CH double bond); 5.42 (m; 2H; H-2 and H-3); 4.75 (t; J 7.7 Hz; 1H; H α); 4.63 (dd; J 4.7 Hz; J 9.6 Hz; 1H; H α); 3.83 (dd; J 2.4 Hz; J 5.6 Hz; 1H; H-4); 3.72 (dd; J 2.7 Hz; J 12.2 Hz; 1H; H-5); 3.66 (dd; J 2.7 Hz; J 12.2 Hz; 1H; H-5′); 3.21 (dd; J 4.9 Hz; J 14.2 Hz; 1H; H′ β); 3.11 (m; 2H; 2×H′ β); 2.97 (dd; J 9.8 Hz; J 14.2 Hz; 1H; H′ β); 1.92 (s; 3H; NHCOCH₃); 1.90 (s; 3H; NHCOCH₃); ¹³C NMR (125.7 MHz; CD₃OD): δ 172.1-172.08 (2×NHCOCH₃); 171.2-170.6 (2×CO); 164.7-151.1 (2×CO uridine); 141.1 (CH double bond); 137.2 to 126.7 (C arom); 102.3 (CH double bond); 86.4 (C-1); 83.8 (C-4); 73.9-72.4 (C-2 and C-3); 61.2 (C-5); 54.3-54.0 (2×CH amino acid); 37.4-36.6 (2×CH₂ amino acid); 21.14-21.12 (2×NHCOCH₃); MS (ES⁺): m/z=645 [M+Na]⁺; HRMS (ES⁺): calculated 640.2619; measured 640.2615 [M+NH₄].

3-O-carboxy-(N-acetyl-L-phenylalanine)-uridine: [α]_{D}²⁵=−14.3 (c=0.82 in methanol); IR νₘₐₓ (KBr): 3310 cm⁻¹ (OH, NH); 1754-1709 cm (C═O); 1544 cm⁻¹ (amide); ¹H NMR (500 MHz; CD₃OD): δ 7.97 (d; J 8.0 Hz; 1H; CH double bond); 7.33-7.23 (m; 5H; H arom); 5.92 (d; J 6.3 Hz; 1H; H-1); 5.75 (d; J 7.9 Hz; 1H; CH double bond); 5.19 (dd; J 3.2 Hz; J 5.4 Hz; 1H; H-3); 4.77 (dd; J 6.9 Hz; J 7.7 Hz; 1H; H′ α); 4.43 (m; 1H; H-2); 3.91 (dd; J 2.7 Hz; J 6.5 Hz; H-4); 3.74 (dd; J 12.1 Hz; J 2.8 Hz; 1H; H-5); 3.66 (dd; J 12.5 Hz; J 2.8 Hz; 1H; H-5′); 3.19 (dd; J6.8 Hz; J 13.6 Hz; 1H; H′ β); 3.07 (dd; J8.4 Hz; J 14.2 Hz; 1H; H′ β); 1.96 (s; 3H; NHCOCH₃); ¹³C NMR (125.7 MHz; CD₃OD): δ 172.2 (NHCOCH₃); 171.2 (CO); 164.8-151.4 (2×CO uridine); 141.2 (CH double bond); 137.1 to 126.7 (C arom); 102.0 (CH double bond); 88.5 (C-1); 83.1 (C-4); 73.9 (C-3); 72.9 (C-2); 61.2 (C-5); 54.3 (CH amino acid); 37.3 (CH₂ amino acid); 21.1 (NHCOCH₃); MS (ES⁺): m/z=456 [M+Na]⁺; HRMS (ES⁺): calculated 451.1829; measured 451.1818 [M+NH₄].

2-O-carboxy-(N-acetyl-L-phenylalanine)-uridine: [α]_{D}²⁵=−14.3 (c=0.82 in methanol); IR νₘₐₓ (KBr): 3310 cm⁻¹ (OH, NH); 1754-1709 cm⁻¹ (C═O); 1544 cm⁻¹ (amide); ¹H NMR (500 MHz; CD₃OD): δ 7.98 (d; J 8.0 Hz; 1H; CH double bond); 7.33-7.23 (m; 5H; H arom); 6.10 (d; J 5.3 Hz; 1H; H-1); 5.73 (d; J 7.9 Hz; 1H; CH double bond); 5.27 (t; J 5.6 Hz; 1H; H-2); 4.75 (dd; J 5.0 Hz; J 9.6 Hz; 1H; H′ α); 4.43 (m; 1H; H-3); 4.05 (dd; J 3.2 Hz; J 7.2 Hz; H-4); 3.87 (dd; J 12.3 Hz; J 2.9 Hz; 1H; H-5); 3.76 (dd; J 12.1 Hz; J 3.3 Hz; 1H; H-5′); 3.28 (dd; J 5.5 Hz; J 13.6 Hz; 1H; H′ β); 2.98 (dd; J 10.0 Hz; J 12.9 Hz; 1H; H′ β); 1.91 (s; 3H; NHCOCH₃); ¹³C NMR (125.7 MHz; CD₃OD): δ 172.18 (NHCOCH₃); 170.7 (CO); 164.8-151.0 (2×CO uridine); 141.6 (CH double bond); 137.1 to 126.7 (C arom); 101.9 (CH double bond); 87.4 (C-1); 85.9 (C-4); 76.5 (C-2); 69.2 (C-3); 61.1 (C-5); 54.1 (CH amino acid); 37.0 (CH₂ amino acid); 21.1 (NHCOCH₃); MS (ES⁺): m/z=456 [M+Na]⁺; HRMS (ES⁺): calculated 451.1829; measured 451.1818 [M+NH₄].

Methyl 5-O-carboxy-(N-acetyl-L-phenylalanine)-β-D-ribofuranoside:

Methyl β-D-ribofuranoside (20 mg; 0.12 mmol), N-acetyl-L-phenylalanine vinyl ester (45 mg; 1.6 eq.) and 20 mg of pH adjusted SBL preparation were suspended in 2 mL of anhydrous pyridine and stirred under nitrogen at 45° C. for 3 weeks. The reaction was then concentrated in vacuo and the residue purified by flash chromatography (chloroform/methanol/acetic acid/water 85/10/0.5/1 v/v) to give after lyophilisation 5′-O-acyl (30 mg; 70%): [α]_{D}²⁵=−25.9 (c=0.23 in methanol); IR νₘₐₓ (KBr): 3421 cm⁻¹ (OH, NH); 1736 cm⁻¹ (C═O); 1654 cm⁻¹ (amide I); 1559 cm⁻¹ (amide II); ¹H NMR (500 MHz; CD₃OD): δ 7.27-7.21 (m; 5H; H arom); 4.75 (s; 1H; H-1); 4.72 (dd; J4.4 Hz; J8.4 Hz; 1H; H a); 4.33 (dd; J2.6 Hz; J 11.0 Hz; 1H; H-5); 4.17 (dd; J 5.1 Hz; J 11.9 Hz; 1H; H-5′); 4.08 (m; 2H; H4 and H-2); 3.89 (d; J 3.8 Hz; 1H; H-3); 3.32 (s; 3H; OCH₃); 3.21 (dd; J 5.0 Hz; J 13.8 Hz; 1H; H′ β); 2.97 (dd; J 9.1 Hz; J 13.1 Hz; 1H; H′ β); 1.91 (s; 3H; NHCOCH₃); ¹³C NMR (125.7 MHz; CD₃OD): δ 173.2-172.8 (2×CO); 138.2-130.2-129.5-127.9 (C arom); 109.9 (C-1); 81.5 (C-4); 75.9 (C-2); 72.7 (C-3); 66.9 (C-5); 55.4-55.3 (OCH₃ and CH amino acid); 38.2 (CH₂ amino acid); 22.2 (NHCOCH₃); MS (ES⁺): m/z=376 [M+Na]⁺; HRMS (ES⁺): calculated 376.1372; measured 376.1370 [M+Na].

5-carboxy-(N-acetyl-L-phenylalanine)-2-deoxyadenosine:

2′-deoxyadenosine (100 mg; 0.40 mmol), N-acetyl-L-phenylalanine vinyl ester (149 mg; 1.6 eq.) and 20 mg of pH adjusted SBL preparation were suspended in 5 mL of anhydrous DMF and stirred under nitrogen at 45° C. for 3 weeks. The reaction was then concentrated in vacuo and the residue purified by flash chromatography (chloroform/methanol/acetic acid/water 100/2/0.5/1 v/v) to give after lyophilisation 22a (10 mg; 6%): [α]_{D}²⁵=+20.0 (c=0.09 in methanol); IR νₘₐₓ (KBr): 3340 cm⁻¹ (OH, NH); 1748 cm⁻¹ (C═O); 1647 cm⁻¹ (amide I); 1602 cm⁻¹ (amide II); ¹H NMR (500 MHz; CD₃OD): δ 8.29 (s; 1H; Hdouble bond); 8.21 (s; 1H; H double bond); 7.27-7.16 (m; 5H; H arom); 6.42 (t; J 6.7 Hz; 1H; H-1); 4.62 (t; J 7.1 Hz; 1H; CH amino acid); 4.36 (dd; J3.6 Hz; J 11.6 Hz; 1H; H-5); 4.33-4.27 (m; 2H; H-5′ and H-3); 4.15 (m; 1H; H-4); 3.07 (dd; J 7.1 Hz; J 13.8 Hz; 1H; CH₂ amino acid); 2.97 (dd; J 8.0 Hz; J 13.8 Hz; 1H; CH₂ amino acid); 2.74 (q; J 6.3 Hz; 1H; H-2); 2.44 (ddd; J 3.8 Hz; J 6.3 Hz; J 13.6 Hz; 1H; H-2′); 2.00 (s; 3H; NHCOCH₃); ¹³C NMR (125.7 MHz; CD₃OD): δ 173.2-173.0 (2×CO); 157.3 (C arm); 153.8 (CH double bond); 141.0 (CH double bond); 137.9-130.2-130.1-129.5-129.2-127.9 (C arom); 85.96 (C-1); 85.93 (C-4); 72.5 (C-3); 65.9 (C-5); 55.7 (CH amino acid); 40.2 (C-2); 38.3 (CH₂ amino acid); 22.2 (NHCOCH₃); MS (ES⁺): m/z=441 [M+H]⁺; HRMS (ES⁺): calculated 441.188643; measured 441.187421 [M+H].

Aminoacylated Dinucleotide, Bz/TBS/DMT-C-P(O(CH₂)₂CN)A-[PheNHBoc]

2/3-O-carboxy-(N-tert-butyloxycarbonyl-L-phenylalanine)-adenosine (100 mg; 1.1 eq.) and the commercial phosphoramidite (170 mg; 0.18 mmol) were solubilised in 2 mL of freshly distilled acetonitrile and stirred 1 h in the presence of molecular sieves. Then 2 mL of the commercial tetrazole solution (0.45 M in acetonitrile) was slowly added; after 3 h at room temperature 1 mL of tetrazole solution was added.

After 1 h, 5 mL of an iodine solution in THF/collidine/water (2/2/1) was slowly added, and the mixture was stirred for 30 minutes at room temperature. Then 10 mL of a solution of sodium thiosulfate was added and stirred 10 minutes. After addition of dichloromethane, the organic layer was separated and dried over magnesium sulfate and concentrated in vacuo and the residue purified by flash chromatography (chloroform pure then 2% methanol) to give 2-O′-acyl (30 mg; 12%) and 3-O′-acyl (73 mg; 30%) as yellow pale solids:

2′: [α]_{D}²⁵=+26.7 (c=0.18 in chloroform); IR νₘₐₓ (KBr): 3446-3372 cm⁻¹ (OH, NH); 2256 cm⁻¹ (CN); 1708 cm (C═O); 1654-1640 cm⁻¹ (amide I); 1508-1483 cm⁻¹ (amide II); ¹H NMR (500 MHz; CDCl₃): δ 8.35 (m; 1H); 7.94-7.76 (m; 4H); 7.60 (m; 1H); 7.50 (m; 2H); 7.40-7.18 (m; 16H); 6.92-6.80 (m; 4H); 6.10-5.65 (m; 2H); 5.64-5.50 (m; 1H); 5.22-5.10 (m; 2H); 4.80-4.70 (m; 2H); 4.70-3.90 (m; 10H); 3.83 (m; 6H; 2×OCH₃); 3.70 (m; 1H); 3.45 (m; 1H); 3.15 (m; 2H); 2.70 (m; 1H); 2.50 (m; 1H); 1.43 (m; 9H; 3×CH₃ Boc group); 0.90 (m; 9H; 3×CH₃—Si); 0.14 (m; 6H; 2×CH₃—Si); ¹³C NMR (125.7 MHz; CDCl₃): δ 171.8-171.7 (2×CO); 162.0 (2×CO); 159.1-158.9 (C arom); 156.0-155.3 (C═C); 153.0-148.6-148.5-144.0-143.9-135.9-127.4 (C arom); 120.9 (C—NH₂); 116.6-116.5 (CN); 113.6 (C═C); 88.6 (1×C); 87.7-87.5 (C DMT group); 86.0-85.5 (2×C); 80.5 (C Boc group); 76.2-75.5-75.1-74.3-74.1 (5×C); 63.3 (m; CH₂); 62.6 (s; CH₂); 55.6-55.5-55.4 (OCH₃); 55.1-54.9 (CH amino acid); 39.1 (CH₂ amino acid); 29.9 (CH₂); 28.5 (CH₃ Boc group); 26.0-25.9-25.8 (Si—C(CH₃)₃); 19.7 (m; CH₂); 18.3-18.4 (Si—C); −4.4; −4.5; −5.2 (2×Si—CH₃); ³¹P NMR (161.9 MHz; CDCl₃): δ −0.214; −3.421; MS (ES⁺): m/z=1415 [M+Na]⁺; HRMS (ES⁺): calculated; measured [M+].

3′: [α]_{D}²⁵=+8.2 (c=0.77 in chloroform); IR νₘₐₓ (KBr): 3339 cm⁻¹ (OH, NH); 2255 cm⁻¹ (CN); 1750-1701 cm⁻¹ (C═O); 1656-1610 cm⁻¹ (amide I); 1509-1485 cm⁻¹ (amide II); ¹H NMR (400 MHz; CDCl₃): 388.22 (m; 1H); 8.14-7.66 (m; 4H); 7.50 (m; 1H); 7.39 (m; 2H); 7.32-7.02 (m; 16H); 6.84-6.72 (m; 4H); β 6.10-5.75 (m; 2H); 5.65 (m; 1H); 5.40-5.20 (m; 2H); 5.00-4.70 (m; 2H); 4.60-3.80 (m; 10H); 3.72 (s; 3H; OCH₃); 3.71 (s; 3H; OCH₃); 3.55 (m; 1H); 3.40 (m; 1H); 3.05 (m; 2H); 2.52 (m; 1H); 2.40 (m; 1H); 1.30 (m; 9H; 3×CH₃ Boc group); 0.78 (m; 9H; 3×CH₃—Si); 0.05 (m; 6H; 2×CH₃—Si); ¹³C NMR (100.6 MHz; CDCl₃): δ 171.5-171.4 (2×CO); 162.7-162.6 (2×CO); 158.7-158.6-158.2-157.1-155.7-155.6-155.0-153.1-152.9-149.2-149.1-147.7-144.8-144.3-143.7-136.0-126.6 (C arom); 119.8-119.7 (C—NH₂); 116.5-116.3 (CN); 113.3-112.9 (C═C); 97.2-89.4-89.2-88.4 (4×C); 87.5-87.4 (C DMT group); 86.3-81.9-81.1 (3×C); 80.6-80.5 (C Boc group); 80.2-79.9-76.1-75.4-75.2-73.2-72.8-72.6-72.4 (9×C); 67.2-66.9 (m; CH₂); 62.4-62.1 (m; CH₂); 55.2-55.1-55.0 (2×OCH₃ and CH amino acid); 37.6 (CH₂ amino acid); 29.6 (CH₂); 28.2 (CH₃ Boc group); 25.5 (Si—C(CH₃)₃); 19.3 (CH₂); 18.0-17.9 (Si—C); −4.7; −4.8; −5.3; −5.34 (2×S₁—CH₃); ³P NMR (161.9 MHz; CDCl₃): δ −1.56; MS (ES⁺): m/z=1415

Aminoacylated Dinucleotide, Bz/TBS/DMT-C-P(O(CH₂)₂CN)A-[PheNHPhAc]

2/3-O-Carboxy-(N-phenylacetamyl-L-phenylalanine)-adenosine (50 mg, 0.094 mmol) and RNA phosphoramidite (82 mg, 0.085 mmol) were stirred in MeCN (2 ml) in the presence of molecular sieves for 1 h. After this time tetrazole (0.45 M in MeCN, 3 ml) was added slowly and the reaction mixture stirred under an atmosphere of argon for two hours. At this point Iodine (0.1 M in THF:H₂O:collidine (2:2:1), 4 ml) was added and the mixture stirred for 30 min after which time Na₂S₂O₃ solution (0.1 M, 10 ml) was added and the mixture stirred for a further 10 min. The reaction mixture was diluted with DCM (30 ml) and the organic phase dried (MgSO₄), filtered and concentrated in vacuo. The residue was purified by flash column chromatography (methanol:CHCl₃, 1:20) to afford a mixture of 2/3-O-linked phenylacetamide protected dinucleotides (54 mg, 45% yield) as a clear oil; m/z (ES⁺): 1433.50 (M+Na⁺, 50), 1411.52 (M+H⁺, 100%); Isotope Distribution calculated for C₇₃H₇₉O₁₆N₁₀SiP (M+H⁺): 1415.54 (3), 1414.53 (18), 1413.53 (48), 1412.53 (92), 1411.53 (100%). Found: 1415.58 (3), 1414.57 (26), 1413.49 (47), 1412.54 (100), 1411.52 (100%).

Partially Deprotected Aminoacylated Dinucleotide, Bz/TBS-C-P(O(CH₂)₂CN)A-[PheNHPhAc]

A mixture of 2/3-O-linked phenylacetamide protected dinucleotides (89 mg, 0.063 mmol) was stirred in nitromethane:methanol:Cl₃CO₂CH (95:5:3, 4 ml) for 30 min, after which time t.l.c. indicated complete conversion of starting material (R_{f} 0.5) to a major product (R_{f} 0.4). The reaction mixture was quenched with NEt₃ (0.5 ml) and diluted with DCM (50 ml). The organic phase was washed with distilled water (30 ml), then dried (MgSO₄), filtered and concentrated in vacuo. The residue was purified by flash column chromatography (methanol:ethyl acetate, 1:25 to 1:10) to afford a mixture of 2/3-O-linked phenylacetamide protected 5′ deprotected dinucleotides (47 mg, 67% yield) as a clear oil; m/z (ES⁺): 1131.44 (M+Na⁺, 60), 1109.41 (M+H⁺, 100%); Isotope Distribution calculated for C₅₂H₆₁O₁₄N₁₀SiP (M+H⁺): 1112.40 (8), 1111.40 (29), 1110.40 (67), 1109.40 (100%). Found: 1112.39 (13), 1111.26 (29), 1110.42 (66), 1109.41 (100%).

4-[N-((1S)-1-carboxy-2-phenylethyl)carbamoyl]butanoic Acid

Glutaric anhydride (0.5 g; 4.4 mmol) and L-phenylalanine (0.73 g; 1 eq.) were dissolved in a mixture THF/DMF (15/5 mL) and stirred for 24 h at 50° C. for 4 h. The mixture was then stirred at room temperature overnight. After evaporation in vacuo the crude product was purified by flash chromatography (chloroform/methanol 9/1 v/v) to give the diacid (930 mg, 76%): [α]_{D}²⁵=+11.6 (c=0.2 in chloroform); IR νₘₐₓ (film): 3302 cm⁻¹ (large, OH acid, NH); 1720 cm⁻¹ (C═O); 1658 cm⁻¹ (amide I); 1552 cm⁻¹ (amide II); ¹H NMR (500 MHz, CD₃OD) δ 7.30-7.22 (m; 5H; H arom); 4.70(dd; J 4.8 Hz; J 9.5 Hz; 1H; H α); 3.24 (dd; J 4.9 Hz; J 13.7 Hz; 1H; H′ β); 2.95 (dd; J 9.7 Hz; J 13.8 Hz; 1H; H′ β); 2.73 (t; J7.3 Hz; 1H); 2.24-2.19 (m; 3H); 1.80 (q; J 7.6 Hz; 1H); ¹³C NMR (125 MHz, CD₃OD) δ 176.8-175.2-174.8 (3×CO); 138.5-127.8 (C arom); 54.9 (CH amino acid); 38.4 (CH₂ amino acid); 35.7-33.9-22.1 (3×CH₂); MS (ES⁺) m/z=302 [M+Na]⁺.

Vinyl-4-{N-[(1S)-2-phenyl-1 (vinyloxy-carbonyl)ethyl]carbamoyl}butanoate 21

A mixture of diacid (0.78 g, 2.8 mmol), vinyl acetate (25 mL, 0.28 mol), palladium acetate (125 mg, 0.56 mmol) and potassium hydroxide (160 mg, 0.28 mmol) was stirred for 24 h at r.t. The mixture was then poured into ether (100 mL) and filtered through a celite bed. After evaporation in vacuo the crude product was purified by flash chromatography (hexane/ethyl acetate 2/1 v/v) to give the acyl donor 21 (270 mg, 30%): [α]_{D}²⁵=+20.5 (c=1.15 in chloroform); IR νₘₐₓ (film): 1751 cm⁻¹ (C═O); 1648 cm⁻¹ (amide I); 1520 cm⁻¹ (amide II); ¹H NMR (250 MHz, CDCl₃) δ 7.28-7.06 (m; 5H; H arom); 6.25(d; J 8.4 Hz; NH); 4.92 (m and dd; J 1.9 Hz; J 13.9 Hz; 2H; H α and H vinyl group); 4.84 (dd; J 1.6 Hz; J 13.9 Hz; 1H; H vinyl group); 4.63 (dd; J 2.0 Hz; J 6.3 Hz; 1H; H vinyl group); 4.54 (dd; J 1.5 Hz; J 6.3 Hz; 1H; H vinyl group); 3.17 (dd; J 5.6 Hz; J 13.8 Hz; 1H; H′ β); 3.04 (dd; J 6.8 Hz; J 13.9 Hz; 1H; H′ β); 2.35 (t; J 7.3 Hz; 1H); 2.22 (t; J 7.3 Hz; 2H); 1.89 (q; J 7.2 Hz; 2H); ¹³C NMR (62.9 MHz, CDCl₃) δ 171.8-170.1-168.9 (3×CO); 140.9-140.7-135.5-129.2-128.6-127.2 (6×C arom); 99.1-97.7 (CH₂ vinyl group); 52.8 (CH amino acid); 37.5-34.7-32.6-20.2 (4×CH₂); MS (ES⁺) m/z=332 [M+H]⁺;354 [M+Na]⁺; HRMS (ES⁺): calculated 332.149798; measured 332.149750 [M+H].

Tethered Monosaccharide 16i

Methyl α-D-mannopyranoside 16a (100 mg; 0.51 mmol), di-vinylester 21 (273 mg; 1.6 eq.) and 30 mg of pH adjusted SBL preparation were suspended in anhydrous pyridine (5 mL) and stirred under nitrogen at 45° C. for 3 weeks. The reaction was filtered through celite, evaporated and the residue purified by flash chromatography (ethyl acetate/methanol 100/1 v/v) to give 16i (66 mg; 27%) after lyophilisation: [α]_{D}²⁵=+30.6 (c=0.17 in methanol); IR νₘₐₓ (KBr): 3332 cm⁻¹ (OH, NH); 1748 cm⁻¹ (CO); 1645 cm⁻¹ (amide I); 1537 cm⁻¹ (amide II); ¹³C NMR (125 MHz, CD₃OD) δ 175.0-172.9-171.6 (3×C═O); 142.3 (CH vinyl ester); 138.3-130.3-129.5-129.4-127.8-127.7 (C arom); 102.8 (C-1); 97.9 (CH₂ vinyl ester); 72.5-71.93-71.89-68.5 (C-2; C-3; C-4; C-5); 65.8 (C-6); 55.3-55.0 (OCH₃ and CH amino acid); 38.4 (CH₂ amino acid); 35.5-33.4-21.7 (3×CH₂); ¹H NMR (500 MHz, CD₃OD) δ 7.28-7.19 (m; 6H; 5H arom and 1H vinyl ester); 4.87 (dd; J 1.6 Hz; J 14.1 Hz; 1H; H vinyl ester); 4.77 (dd; J 4.8 Hz; J 9.6 Hz; 1H; H amino acid); 4.61 (d; J 1.6 Hz; 1H; H-1); 4.57 (dd; J 6.3 Hz; J 1.6 Hz; H vinyl ester); 4.43 (dd; J 11.7 Hz; J 2.2 Hz; 1H; H-6); 4.31 (dd; J 6.2 Hz; J 11.3 Hz; 1H; H-6′); 3.78 (m; 1H; H-2); 3.70-3.63 (m; 3H; H-5; H-4; H-3); 3.36 (s; 3H; OCH₃); 3.25 (dd; J 4.6 Hz; J 13.9 Hz; 1H; H amino acid); 2.93 (dd; J 9.8 Hz; J 14.2 Hz; 1H; H amino acid); 2.25 (m; 2H; CH₂); 2.21 (m; 2H; CH₂); 1.80 (m; 2H; CH₂); MS (ES⁺) m/z=482 [M+H]⁺; 504 [M+Na]⁺; HRMS (ES⁺): calculated 504.1846; measured 504.1843 [M+H].

Enzmatic Acylation of Thiophenyl α-D-mannopyrrannoside with PheNHBoc

S-phenyl-α-D-mannopyrannoside 19a (300 mg; 1.1 mmol), vinyl N-tert-butyloxycarbonyl-L-phenylalaninate (513 mg; 1.6 eq.) and 30 mg of pH adjusted SBL preparation were suspended in 5 mL of anhydrous pyridine and stirred under nitrogen at 45° C. for 3 weeks. The reaction was filtered through celite, evaporated and the residue purified by flash chromatography (chloroform/methanol/acetic acid/water 100/2/0.5/1 then 100/4/0.5/1 v/v) to give after lyophilisation, 3 regioisomers:

S-Phenyl-3-O-carboxy-(N-tert-butyloxycarbonyl-L-phenylalanine)-α-D-mannopyranoside 19i: (75 mg, 13%): [α]_{D}²⁵=+87.9 (c=0.39 in methanol); IR νₘₐₓ (KBr): 3392 cm⁻¹ (OH, NH); 1742 cm⁻¹ (CO); 1648 cm⁻¹ (amide I); 1522 cm⁻¹ (amide II); ¹³C NMR (100 MHz, CD₃OD) δ 173.1 (C═O); 157.9-138.4-135.3-133.1-133.0-130.4-130.1-129.4-128.7-127.8 (C arom); 90.1 (C-1); 80.7 (C Boc); 76.9 (C-3); 75.7 (C-5); 71.2 (C-2); 65.8 (C-4); 62.3 (C-6); 56.6 (CH amino acid); 38.6 (CH₂ amino acid); 28.7 (3×CH₃); ¹H NMR (500 MHz, CD₃OD) δ 7.60-7.50 (m; 2H; H arom); 7.38-7.19 (m; 8H; H arom); 5.46 (s; 1H; H-1); 5.06 (dd; J 3.1 Hz; J 9.4 Hz; 1H; H-3); 4.50 (dd; J 4.8 Hz; J 9.2 Hz; 1H; H amino acid); 4.25 (m; 1H; H-2); 4.21-4.15 (m; 1H; H-5); 4.06 (t; J 9.9 Hz; 1H; H-4); 3.87-3.83 (m; 2H; H-6 and H-6′); 3.28 (dd; J 5.3 Hz; J 14.0 Hz; 1H; H amino acid); 2.97 (dd; J 9.7 Hz; J 14.5 Hz; 1H; H amino acid); 1.40 (s; 9H; 3×CH₃); MS (ES⁺) m/z=542 [M+Na]⁺; HRMS (ES⁺): calculated 542.1825; measured 542.1815 [M+Na].

S-Phenyl-2-O-carboxy-(N-tert-butyloxycarbonyl-L-phenylalanine)-α-D-mannopyranoside 19h: (67 mg, 12%): [α]_{D}²⁵=+49.4 (c=0.42 in methanol); IR νₘₐₓ (KBr): 3413 cm⁻¹ (OH, NH); 1748 cm⁻¹ (CO); 1692 cm⁻¹ (amide I); 1584 cm⁻¹ (amide 11); ¹³C NMR (100 MHz, CD₃OD) δ 173.0 (C═O); 157.9-138.2-135.2-133.2-130.4-130.1-129.4-128.9-127.8 (C arom); 87.4 (C-1); 80.7 (C Boc); 76.3 (C-2); 75.9 (C-5); 71.5 (C-3); 69.0 (C4); 62.5 (C-6); 56.5 (CH amino acid); 38.6 (CH₂ amino acid); 28.7 (3×CH₃); ¹H NMR (500 MHz, CD₃OD) δ 7.56-7.51 (m; 2H; H arom); 7.38-7.13 (m; 8H; H arom); 5.32 (s; 1H; H-1); 5.30 (m; 1H; H-2); 4.47 (dd; J 5.3 Hz; J 8.3 Hz; 1H; H amino acid); 4.08 (m; 1H; H-5); 3.93-3.85 (m; 2H; H-6 and H-3); 3.83-3.74 (m; 2H; H-6′ and H-4); 3.19 (dd; J 5.6 Hz; J 13.9 Hz; 1H; H amino acid); 2.94 (dd; J 8.8 Hz; J 13.7 Hz; 1H; H amino acid); 1.39 (s; 9H; 3×CH₃); MS (ES⁺) m/z=[M+Na]⁺; HRMS (ES⁺): calculated; measured [M+].

S-Phenyl-6-O-carboxy-(N-tert-butyloxycarbonyl-L-phenylalanine)-α-D-mannopyranoside 19g: (91 mg, 16%): [α]_{D}²⁵=+111.1 (c=0.59 in methanol); IR νₘₐₓ (film): 3372 cm⁻¹ (OH, NH), 1741 cm⁻¹ (C═O), 1717 cm (amide I), 1696 cm⁻¹ (amide II); ¹³C NMR (125 MHz, CD₃OD) δ 173.4 (C═O); 157.7-138.2-135.6-132.7-130.4-130.3-130.1-130.0-129.4-128.5-127.7 (C arom); 90.1 (C-1); 80.6 (C Boc); 73.5 (C-2); 73.07-72.99-68.9 (C-3; C-4; C-5); 65.8 (C-6); 56.2 (CH amino acid); 38.6 (CH₂ amino acid); 28.6 (3×CH₃); ¹H NMR (500 MHz, CD₃OD) δ 7.52 (m; 2H; H arom); 7.22 (m; 8H; H arom); 5.49 (s; 1H; H-1); 4.49 (m; 1H; H-6); 4.41 (dd; J 4.8 Hz; J 9.4 Hz; 1H; H amino acid); 4.31 (m; 2H; H-6′ and H-5); 4.14 (m; 1H; H-2); 3.73 (m; 2H; H-3 and H-4); 3.09 (dd; J 4.5 Hz; J 13.9 Hz; 1H; H amino acid); 2.83 (dd; J 9.0 Hz; J 14.1 Hz; 1H; H amino acid); 1.38 (s; 9H; 3×CH₃); MS (ES⁺) m/z=542 [M+Na]⁺; 1061 [2M+Na]⁺; HRMS (ES⁺): calculated 537.2271; measured 537.2273 [M+NH₄].

Glycosylation with 19g

S—Phenyl-6-O-carboxy-(N-tert-butyloxycarbonyl-L-phenylalanine)-α-D-mannopyranoside 19g (50 mg; 0.01 mmol) and 1,2-3,4-Di-O-isopropylidene galactopyranoside (commercial; 21 mg; 1 eq.) were dissolved in freshly distilled acetonitrile (4 mL) and the mixture was cooling down at 0° C. (ice bath). Then N-iodosuccinimide (25 mg; 1.15 eq.) and triethylsilyl triflate (24 μL in 0.5 mL acetonitrile) were slowly added. After 3 h, the reaction was concentrated under vacuo and the residue purified by flash chromatography (chloroform/methanol 100/2 v/v) to give the corresponding disaccharide 57 (15 mg; 28%) as a mixture of anomeres α/β 3/2: [α]_{D}²⁵=−24.3 (c=0.11 in methanol); IR νₘₐₓ (KBr): 3436 cm⁻¹ (OH, NH); 1716 cm⁻¹ (CO); 1514 cm⁻¹ (amide); ¹³C NMR (125 MHz, CD₃OD) δ 176.1-171.8-171.2 (C═O); 154.4-134.9-128.4-128.3-127.7-127.6-126.1-125.9 (C arom); 108.44-108.41-107.8-107.7 (C isopropylidene); 99.4-98.6-95.3-95.2 (C-1; C-1′a and C-1′β); 79.3 (C Boc); 72.9-72.5-70.3-70.2-70.0-69.6-69.5-69.49-69.3-69.2-69.1-68.0-67.0-66.9-66.5-65.4-65.3-63.3-62.9 (C-2; C-3; C4; C-5; C-6; C-2′; C-3′; C-4′; C-5′; C-6′); 53.7-53.4 (CH amino acid); 36.8 (CH₂ amino acid); 28.7-28.6 (CH₃ Boc); 25.1-25.0-24.9-24.8-23.9-23.8-23.4-23.2 (CH₃ isopropylidene); ¹H NMR (500 MHz, CD₃OD) δ 2H); 4.78 (d; J 0.5 Hz; 1H); 4.53 (dd; J 2.5 Hz; J 7.4 Hz; 1H); 4.47 (m; 1H; H amino acid); 4.35 (m; 1H); 4.28 (m; 1H); 4.24 (dd; J 2.5 Hz; J 5.1 Hz; 1H); 4.12 (d; J 8.0 Hz; 1H); 4.03 (m; 1H); 3.98 (m; 1H); 3.87 (m; 2H); 3.74 (m; 2H); 3.60 (dd; J 5.5 Hz; J 10.3 Hz; 1H); 3.52 (m; 1H); 3.42 (dd; J 2.8 Hz; J 8.8 Hz; 1H); 3.34 (m; 1H); 3.07 (dd; J 5.7 Hz; J 13.5 Hz; 1H; CH₂ amino acid); 3.00 (dd; J 5.7 Hz; J 13.5 Hz; 1H; CH₂ amino acid); 1.45-1.35-1.34-1.26-1.25-1.20-1.19 (CH₃ isopropylidene); MS (ES⁺) m/z=692 [M+Na]⁺; HRMS (ES⁺): calculated 692.2894; measured 692.2893 [M+Na].

Enzymatic Acylation of Thiophenyl α-D-mannopyrannoside with PheNHPhAc

S-phenyl-α-D-mannopyrannoside 19a (300 mg; 1.1 mmol), vinyl N-phenylacetyloxycarbonyl-L-phenylalaninate (545 mg; 1.6 eq.) and 30 mg of pH adjusted SBL preparation were suspended in 5 mL of anhydrous pyridine and stirred under nitrogen at 45° C. for 3 weeks. The reaction was filtered through celite, evaporated and the residue purified by flash chromatography (ethyl acetate/methanol 1% v/v) to give after lyophilisation, 3 regioisomers: S-Phenyl-2,6-Di-O-carboxy-(N-phenylacetyloxycarbonyl-L-phenylalanine)-α-D-mannopyranoside 19f: (62 mg, 7%): [α]_{D}²⁵=+38.4 (c=0.37 in methanol); IR νmax (KBr): 3312 cm⁻¹ (OH, NH); 1740 cm⁻¹ (CO); 1648 cm⁻¹ (amide I); 1533 cm⁻¹ (amide II); ¹³C NMR (100 MHz, CD₃OD) δ 173.8-173.7-172.6-172.2 (4×C═O); 137.8 to 127.8 (C arom); 87.0 (C-1); 76.2 (C-2); 73.1 (C-5); 71.3 (C-3); 69.1 (C-4); 65.7 (C-6); 55.2-54.9 (2×CH amino acid); 43.39-43.37 (2×CH₂Ph); 38.1 (2×CH₂ amino acid); ¹H NMR (400 MHz, CD₃OD) δ 7.52-7.47 (m; 2H; H arom); 7.31-7.07 (m; 2H; H arom); 7.05-7.00 (m; 2H; H arom); 5.32 (m; 2H; H-1 and H-2); 4.82 (dd; J 5.7 Hz; J 8.3 Hz; 1H; H amino acid); 4.71 (dd; J 4.9 Hz; J 8.9 Hz; 1H; H amino acid); 4.50 (m; 1H; H-6); 4.34 (m; 2H; H-5 and H-6′); 3.91 (dd; J 2.9 Hz; J 9.5 Hz; 1H; H-3); 3.73 (t; J 9.5 Hz; 1H; H-4); 3.50 (s; 2H; CH₂Ph); 3.42 (d; J 4.3 Hz; 2H; CH₂Ph); 3.18 (dd; J 5.7 Hz; J 13.8 Hz; 1H; H amino acid); 3.11 (dd; J 4.9 Hz; J 14.1 Hz; 1H; H amino acid); 3.03 (dd; J 8.3 Hz; J 13.2 Hz; 1H; H amino acid); 2.88 (dd; J 9.2 Hz; J 14.1 Hz; 1H; H amino acid); MS (ES⁺) m/z=803 [M+H]⁺; 825 [M+Na]⁺; HRMS (ES⁺): calculated 825.2822; measured 825.2831 [M+Na].

S—Phenyl-3-O-carboxy-(N-phenylacetyloxycarbonyl-L-phenylalanine)-α-D-mannopyranoside 19e:

(46 mg, 8%): [α]_{D}²⁵=+84.7 (c=0.23 in methanol); IR νₘₐₓ (KBr): 3392 cm⁻¹ (OH, NH); 1742 cm⁻¹ (CO); 1648 cm⁻¹ (amide I); 1522 cm⁻¹ (amide II); ¹³C NMR (100 MHz, CD₃OD) δ 173.9-172.42 (2×C═O); 138.0 to 127.8 (C arom); 90.1 (C-1); 77.0 (C-3); 75.8 (C-5); 71.2 (C-2); 65.8 (C-4); 62.3 (C-6); 55.2 (CH amino acid); 43.4 (CH₂Ph); 38.1 (CH₂ amino acid); ¹H NMR (400 MHz, CD₃OD) δ 7.59-7.51 (m; 2H; H arom); 7.38-7.11 (m; 13H; H arom); 5.47 (d; J 1.6 Hz; 1H; H-1); 5.08 (dd; J 3.4 Hz; J 9.8 Hz; 1H; H-3); 4.84 (dd; J 4.8 Hz; J 8.6 Hz; 1H; H amino acid); 4.25 (dd; J 1.6 Hz; J 3.1 Hz; 1H; H-2); 4.20-4.13 (m; 1H; H-5); 4.04 (t; J 9.8 Hz; 1H; H-4); 3.88-3.78 (m; 2H; H-6 and H-6′); 3.51 (s; 2H; CH₂Ph); 3.32 (dd; J 5.0 Hz; J 13.3 Hz; 1H; H amino acid); 3.09 (dd; J 8.9 Hz; J 13.3 Hz; 1H; H amino acid); MS (ES⁺) m/z=560 [M+Na]⁺; HRMS (ES⁺): calculated 560.1719; measured 560.1718 [M+Na]. S—Phenyl-6-O-carboxy-(N-phenylacetyloxycarbonyl-L-phenylalanine)-α-D-mannopyranoside 19d: (172 mg, 29%): [α]_{D}²⁵=+110.3 (c=0.36 in methanol); IR νₘₐₓ (KBr): 3312 cm⁻¹ (OH, NH); 1740 cm⁻¹ (CO); 1648 cm⁻¹ (amide I); 1533 cm⁻¹ (amide II); ¹³C NMR (100 MHz, CD₃OD) δ 174.3-173.1 (2×C═O); 138.4 to 128.2 (C arom); 90.6 (C-1); 73.9-73.52-73.47-69.3 (C-2; C-3; C-4; C-5); 66.3 (C-6); 55.3 (CH amino acid); 43.9 (CH₂Ph); 38.5 (CH₂ amino acid); ¹H NMR (400 MHz, CD₃OD) δ 7.55-7.48 (m; 2H; H arom); 7.29-7.03 (m; 13H; H arom); 5.50 (d; J 1.2 Hz; 1H; H-1); 4.74 (dd; J 4.7 Hz; J 9.1 Hz; 1H; H amino acid); 4.47 (m; 1H; H-6); 4.37 (m; 1H; H-6′); 4.34-4.27 (m; 1H); 4.14 (m; 1H; H-2); 3.76-3.72 (m; 2H); 3.47 (d; J 5.6 Hz; 2H; CH₂Ph); 3.12 (dd; J 4.5 Hz; J 14.3 Hz; 1H; H amino acid); 2.87 (dd; J 9.0 Hz; J 13.5 Hz; 1H; H amino acid); MS (ES⁺) m/z=560 [M+Na]⁺; HRMS (ES⁺): calculated 560.1719; measured 560.1729 [M+Na].

Diethyl Squarate Mono-Amide

Phenyl 6-O-carboxy-(N-tert-butyloxycarbonyl-L-phenylalanine)-1-thio-α-D-mannopyranoside (22 mg, 0.043 mmol) was stirred in a solution of DCM:TFA (9:1, 2 ml) with triethylsilane (14 μl, 0.084 mmol) for 25 min when t.l.c. (methanol:CHCl₃, 1:4) indicated complete conversion of starting material (R_{f} 0.5) to a single product (R_{f} 0.3). The reaction mixture was concentrated in vacuo (toluene) to give 24 and the residue dissolved in a solution of DCM:NEt₃ (9:1, 1 ml). Diethyl squarate (32 μl, 0.215 mmol) was added and the mixture stirred for 6 h 30 min at which point t.l.c. (methanol:CHCl₃, 1:4) indicated complete conversion of starting material (R_{f} 0.3) to a single product (R_{f} 0.6). The reaction mixture was concentrated in vacuo (toluene) and the residue purified by flash column chromatography (methanol:ethyl acetate, 3:97) to afford mono-amide (18 mg, 76% yield) as a clear oil; [α]_{D}²⁵+80.9 (c, 1.74 in CHCl₃); vₘₐₓ (CHCl₃, thin-film): 3396 (br, OH/NH), 1744, 1704 (st, C═O), 1599 (N—CO/C═C) cm⁻¹; δ_{H} (400 MHz+COSY, CDCl₃): 1.26-1.29 (3H, m, CH₃CH₂O), 2.73 (IH, s, OH), 2.96 (1H, a-t, J 10.5 Hz, CH₂), 3.26 (1H, d, J 19.4 Hz, CH₂′), 3.85 (1H, s, H-3), 3.90 (1H, d, J 7.5 Hz, H-4), 4.23 (1H, s, H-2), 4.28 (1H, d, J 9.2 Hz, H-5), 4.34-4.75 (5H, m, H-6, H-6′, CH, CH₃CH₂O), 5.51 (1H, s, H-1), 7.09-7.11 (2H, m, Ar), 7.21-7.27 (5H, m, Ar), 7.41-7.43 (2H, m, Ar), 7.80 (1H, d, J 8.6 Hz, Ar); δ_{C} (100 MHz+DEPT, CDCl₃): 15.6 (q, CH₃CH₂O), 39.1 (t, CH₂), 57.2 (d, CH), 64.6 (t, CH₃CH₂O), 70.2 (t, C-6), 67.3, 70.9, 72.0, 72.1 (4×d, C-2, C-3, C-4, C-5), 87.9 (d, C-1), 127.3, 127.5, 128.6, 129.1, 129.4, 131.3 (6×d, CH—Ar), 133.7, 135.6 (2×s, C—Ar), 169.9, 171.8, 177.9, 182.9, 189.2 (5×s, 3×CO, HNC═COEt); m/z (ESI⁻): 542.02 (M−H⁺, 18), 366.96 (93), 286.86 (100%); HRMS calculated for C₂₇H₂₈O₉NS (M−H⁺) 542.1485. Found 542.1478.

Methyl 6-O-carboxy-(L-phenylalanine)-α-D-mannopyranoside 21

Methyl-6-O-carboxy-(N-tert-butyloxycarbonyl-L-phenylalanine)-α-D-mannopyranoside (54 mg, 0.122 mmol) was stirred in a solution of DCM:TFA (19:1, 2 ml) with triethylsilane (39 ul, 0.245 mmol) for 7 h, when t.l.c. (methanol:CHCl₃, 1:4) indicated complete conversion of starting material (R_{f} 0.5) to a single product (R_{f} 0.2). The reaction mixture was concentrated in vacuo (toluene) and the residue purified by flash column chromatography (methanol:ethyl acetate, 1:9 (+0.5% NEt₃)) to afford methyl 6-O-carboxy-(L-phenylalanine)-α-D-mannopyranoside 21 (36 mg, 86% yield) as a clear oil.

Diethyl Squarate Tethered Carbohydrate-To-Carbohydrate System 25

Diethyl squarate mono-amide (100 mg, 0.18 mmol) and amine 21 (125 mg, 0.37 mmol) were stirred in a solution of DCM:NEt₃ (9:1, 2 ml) for 18 h when t.l.c. (methanol:CHCl₃, 1:4) indicated formation of product (R_{f}'S 0.4). The reaction mixture was concentrated in vacuo and the residue purified by flash column chromatography (methanol:ethyl acetate, 1:20 to 1:10) to yield (96 mg).

### REFERENCES

(1) Bashir, N. B.; Phythian, S. J.; Reason, A. J.; Roberts, S. M. J. Chem. Soc. Perkin Trans 1 1995, 2203-2222.

(2) Riva, S.; Chopineau, J.; Kieboom, A. P. G.; Klibanov, A. M. J. Am. Chem. Soc. 1988, 110, 584-589.

(3) Lloyd, R. C.; Dickman, M.; Jones, J. B. Tetrahedron: Asymmetry 1998, 9, 551-561.

(4) Khuntaveepom, K.; DeSantis, G.; Jones, J. B. Tetrahedron: Asymmetry 1999, 10, 2563.

(5) Miyanaga, M.; Ohmori, M.; Imamura, K.; Sakiyama, T.; Nakanishi, K. J: Biosci. Bioeng. 2000, 90, 43.

(6) Waldmann, H.; Heuser, A.; Schulze, S. Tetrahedron Letters 1996, 37, 8725-8728.

(7) Nudelman, A.; Bechor, Y.; Falb, E.; Fisher, B.; Wexler, B. A.; Nudelman, A. Synthetic Communications 1998, 28, 471-474.

(8) Veeneman, G. H.; van Leeuwen, S. H.; van Boom, J. H. Tetrahedron Letters 1990, 31, 1331-1334.

(9) Konradsson, P.; Udodong, U. E.; Fraser-Reid, B. Tetrahedron Letters 1990, 31, 4313-4316.

(10) Hanessian, S. Chem. Rev. 2000, 100, 4443-4463.

(11) Oliver, J. S.; Oyelere, A. J. Org Chem. 1996, 61, 4168-4171.

(12) Montero, J. L.; Criton, M.; Dewynter, G. F.; Imbach, J. L. Tetrahedron Letters 1991, 32, 5357-5358.

(13) Moris, F.; Gotor, V. J. Org. Chem. 1993, 58, 653-660.

(14) Ciuffreda, P.; Casati, S.; Santaniello Bioorg. Med. Chem. Lett. 1999, 9.

(15) Gotor, V.; Moris, F. Synthesis 1992, 626-628.

(16) Wong, C.-H.; Chen, S.-T.; Hennen, W. J.; Bibbs, J. A.; Wang, Y.-F.; Liu, J. L.-C.; Pantoliano, M. W.; Whitlow, M.; Bryan, P. N. J. Am. Chem. Soc. 1990, 112, 945-953.

(17) Ferrero, M.; Gotor, V. Chem. Rev. 2000,100,4319-4347.

(18) Prasad, A. K.; Wengel, J. Nucleosides & Nucleotides 1996, 15, 1347-1359.

(19) Reese, C. B.; Trentham, D. R. Tetrahedron Letters 1965, 29, 2467-2472.

(20) Barone, A. D.; Tang, J.-Y.; Caruthers, M. H. Nucleic Acids Res. 1984, 12, 4051-4061.

(21) Hwu, J. R.; Jain, M. L.; Tsay, S.-C.; Halidmelahi, H. Tetrahedron Letters 1996, 37, 2035-2038.

(22) Saito, H.; Suga, H. J. Am. Chem. Soc. 2001, 123, 7178-7179.

(23) Tennant-Eyles, R. J.; Davis, B. G.; Fairbanks, A. J. Chem. Commun 1999, 11, 1037-1038.

(24) Jung, K. H.; Muller, M.; Schmidt, R. R. Chem. Rev. 2000, 100,4423.

(25) KhmeInitsky J. Am. Chem. Soc. 1997, 119, 11554.

(26) B. G. Davis, J. Chem. Soc., Perkin Trans. 1 1999, 3215-3237

(27) C. M. Taylor, Tetrahedron, 1998, 54, 11317

(28) A. Varki, Glycobiology 1993, 3, 97; R. A. Dwek, Chem. Rev. 1996, 96, 683.

(29) G. F. Springer, J. Molec. Med., 1997, 75, 594.

(30) T. Tsuda and S.-I. Nishimura, J. Chem. Soc., Chem. Commun., 1996, 2779

(31) O. Seitz, ChemBioChem, 2000, 1,215; M. Meldal, P. M. St Hilaire, Curr. Opin. Chem. Biol., 1997, 1, 552.

(32) R. J. Tennant-Eyles and A. J. Fairbanks, Tetrahedron: Asymm., 1999, 10, 391.

(33) M. Dickman, R. C., Lloyd, J. B. Jones, Tetrahedron: Asymm., 1998, 9, 4099.

(34) M. Dickman, R. C., Lloyd, J. B. Jones, Tetrahedron: Asymm., 1998, 9, 551.

(35) M. Lobell, and M. P. Schneider, Synthesis, 1994, 375

(36) P. Sears P, M. Schuster, P. Wang, K. Witte, C.-H. Wong, J. Am. Chem. Soc., 1994, 116, 6521.

(37) B. G. Davis, J. Chem. Soc., Perkin Trans. 1 2000, 2137.

(38) S. J. Chung, S. Takayama, C.-H. Wong, Bioorg Med. Chem. Lett., 1998, 8, 3359.

(39) M. Miyanaga, M. Ohmori, K. Imamura, T. Salciyama and K. Nakanishi, J. Biosci. Bioeng., 2000, 90, 43.

(40) K. Matsumoto, B. G. Davis and J. B. Jones, Chem. Commun., 2001, 903-904.

1. A method of producing an ester linked carbohydrate-peptide conjugate comprising:
(a) providing a vinyl ester amino acid group, and
(b) reacting the vinyl ester amino acid with a carbohydrate acyl acceptor in the presence of an enzyme, to produce thereby an ester-linked carbohydrate-peptide conjugate.

2. A method according to claim 1 wherein step (a) comprises protecting the amine group of the amino acid with a protecting group, reacting the amino acid with vinyl acetate to produce thereby the vinyl ester amino acid group.

3. A method according claim 1 or claim 2, wherein the vinyl ester amino acid group is vinyl ester phenylalanine.

4. A method according to claim 1, wherein the vinyl ester amino acid group is vinyl ester glutamic acid or vinyl ester aspartic acid.

5. A method according to claim 1, wherein the amino acid is extended by terminal chain extension.

6. A method according to claim 5, wherein the amino acid is extended by the addition of a peptide.

7. A method according to claim 1, wherein the acyl carbohydrate acceptor is unprotected.

8. A method according to claim 1, wherein the carbohydrate is selected from the group consisting of mannoses, glycoses, galactases, and N-acetyl glucose.

9. A method according to claim 8, wherein the carbohydrate acyl acceptor has an O-1 substituent.

10. A method according to claim 1, wherein the carbohydrate acyl acceptor comprises a thioglycoside or selenoglycoside.

11. A method according to claim 1, wherein the carbohydrate acyl acceptor is D-mannose.

12. A method according to claim 1, wherein the carbohydrate acyl acceptor is a ribonucleotide.

13. A method according to claim 1, wherein the carbohydrate acyl acceptor is selected to provide a desired conjugation regioselectivity, such as 6-O regioselectivity.

14. A method according to claim 1, further comprising the step of sugar reducing end extension.

15. A method according to claim 1, wherein the enzyme is selected from the group consisting of proteases, lipases, esterases and acylases.

16. A method according to claim 15 wherein the protease is a serine protease.

17. A method according to claim 16 wherein the protease is subtilisin of bacillus lentis.

18. A method according to claim 15, wherein the protease is thermolysin.

19. A method according to claim 1, wherein the vinyl ester amino acid comprises more than one vinyl ester group.

20. A method according to claim 19, wherein carbohydrate acyl donors are conjugated to each of the vinyl ester groups of the vinyl ester amino acid.

21. A method according to claim 20, wherein different carbohydrate acyl donors are conjugated to each vinyl ester group.

22. A method according to claim 1, which comprises generating two or more ester linked carbohydrate-peptide conjugates and then linking the conjugates.

23. A method according to claim 22, wherein the carbohydrate-peptide conjugates are linked by reacting the two conjugates with diethyl squarate.

24. A method according claim 1, further comprising formulating the carbohydrate-peptide conjugate with a pharmaceutically acceptable carrier.

25. A carbohydrate peptide conjugate obtainable by the method of claim 1.
